(19) United States
(12) Patent Application Publication
(10) Pub. No.: US 2011/0203649 A1
(43) Pub. Date: Aug. 25, 2011

(54) USE OF INDANTHRENE COMPOUNDS IN ORGANIC PHOTOVOLTAICS

(75) Inventors: Martin Könemann, Mannheim (DE); Regina Höh, Ludwigshafen (DE); Jae Hyung Hwang, Viernheim (DE); Christian Dörr, Niederkirchen (DE); JianQiang Qu, Shanghai (CN); Gerd Weber, Bad Duerkheim (DE); Gabriele Mattern, Schifferstadt (DE); Peter Erk, Frankenthal (DE)

(73) Assignee: BASF SE, Ludwigshafen (DE)

(21) Appl. No.: 13/029,571

(22) Filed: Feb. 17, 2011

Related U.S. Application Data
(60) Provisional application No. 61/305,977, filed on Feb. 19, 2010.

(51) Int. Cl. H01L 51/46; H01L 31/06; C07D 241/38

(52) U.S. Cl. 136/255; 136/263; 544/338

(57) ABSTRACT

The present invention relates to an organic solar cell which comprises at least one photoactive region which comprises at least one indanthrene compound which is in contact with at least one fullerene compound, and to the use of indanthrene compounds in organic photovoltaics, especially in the form of a component cell of a tandem cell.

### SUBJECT MATTER OF THE INVENTION

The present invention relates to an organic solar cell which comprises at least one photoactive region which comprises at least one indanthrene compound which is in contact with at least one fullerene compound, and to the use of indanthrene compounds in organic photovoltaics.

### BACKGROUND OF THE INVENTION

Owing to diminishing fossil raw materials and the CO₂ which is formed in the combustion of these raw materials and is active as a greenhouse gas, direct energy generation from sunlight is playing an increasing role. “Photovoltaics” is understood to mean the direct conversion of radiative energy, principally solar energy, to electrical energy.

In contrast to inorganic solar cells, the light does not directly generate free charge carriers in organic solar cells, but rather excitons are formed first, i.e. electrically neutral excited states in the form of electron-hole pairs. These excitons can be separated only by very high electrical fields or at suitable interfaces. In organic solar cells, sufficiently high fields are unavailable, and so all existing concepts for organic solar cells are based on exciton separation at photoactive interfaces (organic donor-acceptor interfaces or interfaces to an inorganic semiconductor). For this purpose, it is necessary that excitons which have been generated in the volume of the organic material can diffuse to this photoactive interface. The diffusion of excitons to the active interface thus plays a critical role in organic solar cells. In order to make a contribution to the photocurrent, the exciton diffusion length in a good organic solar cell must at least be in the order of magnitude of the typical penetration depth of light, in order that the predominant portion of the light can be utilized. The efficiency of an organic solar cell is characterized by its open-circuit voltage Voc. Further important characteristics are the short-circuit current I_{SC}, the fill factor FF and the resulting efficiency η.

The first organic solar cell with an efficiency in the percent range was described by Tang et al. in 1986 (C W. Tang et al., Appl. Phys. Lett. 48, 183 (1986)). It consisted of a two-layer system with copper phthalocyanine (CuPc) as the donor material (p-semiconductor) and perylene-3,4:9,10-tetracarboxylic acid bisimidazole (PTCBI) as the acceptor material (n-semiconductor).

A current aim in organic photovoltaics is to provide a new generation of solar cells which are significantly less expensive than solar cells composed of silicon or other inorganic semiconductors such as cadmium indium selenide or cadmium telluride. For this purpose, there is additionally a need for suitable semiconductive light-absorbing materials. One means of absorbing a large amount of light and of achieving good efficiencies is to use a pair of semiconductor materials which are complementary with regard to light absorption, for example comprising a short-wave-absorbing n-semiconductor and a long-wave-absorbing p-semiconductor. This concept is also the basis of the aforementioned first organic solar cell, known as the Tang cell. Even though many fullerene compounds absorb the light only weakly, it has been found that efficient solar cells can be produced when fullerenes or fullerene derivatives, such as C60 or C72, are used as n-semiconductors. It is additionally known, when using weakly absorbing semiconductor materials, to build two solar cells one on top of another. In that case, one cell comprises a combination of the weakly absorbing semiconductor with a semiconductor complementary thereto, which absorbs the short-wave radiation, and the other cell a combination of the weakly absorbing semiconductor with a semiconductor complementary thereto, which absorbs the long-wave radiation. For such tandem cells for combination with fullerenes or fullerene derivatives, two suitable p-semiconductors are required, one of which absorbs the short-wave radiation and one the long-wave radiation. The discovery of suitable semiconductor combinations is not trivial. In tandem cells, the open-circuit voltages Voc of the individual components are additive. The total current is limited by the component cell with the lowest short-circuit current Isc. The two semiconductor materials of the individual cells thus have to be adjusted exactly with respect to one another. There is therefore a great need for p-semiconductive organic absorber materials with long-wave absorption for use in organic solar cells in combination with fullerenes or fullerene derivatives, and especially in tandem cells, with high open-circuit voltage and acceptable short-circuit current.

The first indanthrene dye, Indanthrene Blue RS, was synthesized in 1901 by Bohn at BASF, and was the first representative of the anthraquinone vat dyes. Indanthrene is also referred to as indanthrone or C.I. Pigment Blue 60.

JP 5102506 A describes a photovoltaic cell which has a photoactive region, in which a layer which comprises an organic donor material is in contact with a layer which comprises an organic acceptor material. The photoactive region comprises at least one indanthrene dye and/or an anthraquinoneacridone dye, but exclusively as an electron acceptor (n-semiconductor, electron conductor). Suitable indanthrene dyes described are dyes of the general formula (A)

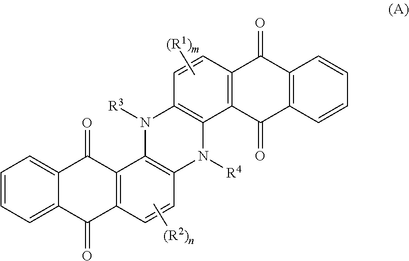

in which

m and n are each 0 to 4,

R¹ and R² are each halogen, alkyl, alkoxy, hydroxyl, amino, acetyl, carboxyl, nitro or cyano, and

R³ and R⁴ are each hydrogen or alkyl.

Suitable organic electron donors described in this document are various phthalocyanines, and polymers with conjugated π-systems, such as polyacetylenes.

It has now been found that, surprisingly, indanthrene compounds are advantageously suitable as electron donors (p-semiconductors, hole conductors) in organic photovoltaics. They are especially suitable for a combination with at least one fullerene compound, such as C60, as an electron acceptor (n-semiconductor, electron conductor). It has especially been found that indanthrene compounds are suitable for use in tandem cells, since they have a long-wave absorption and exhibit a high open-circuit voltage in combination with a fullerene compound, such as C60.

### SUMMARY OF THE INVENTION

The invention firstly provides an organic solar cell comprising at least one photoactive region which comprises at least one indanthrene compound which is in contact with at least one fullerene compound, wherein the indanthrene compound is selected from compounds of the general formula (I)

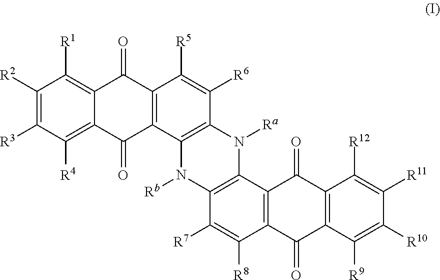

in which
R^{a} and R^{b} are each independently selected from hydrogen, deuterium, unsubstituted or substituted alkyl, unsubstituted or substituted cycloalkyl and unsubstituted or substituted aryl,
the R¹ to R¹² radicals are each independently selected from hydrogen, halogen, nitro, cyano, hydroxyl, carboxyl, carboxylates, SO₃H, sulfonate, Ne^{a}E^{b}, and in each case unsubstituted or substituted alkyl, alkoxy, alkylthio, cycloalkyl, aryl, aryloxy, arylthio, hetaryl, hetaryloxy, hetarylthio, oligo(het)aryl, oligo(het)aryloxy and oligo(het)alkylthio, where E^{a} and E^{b} are each independently hydrogen, alkyl, cycloalkyl or aryl.

The invention further provides for the use of a compound of the general formula (I) as defined above and hereinafter as an electron donor (p-semiconductor, hole conductor) in organic photovoltaics.

The invention further provides novel compounds of the formula I, i.e. compounds of the formula I in which R¹ to R¹² are all hydrogen and R^{a} and R^{b} are both deuterium.

The invention further provides novel compounds of the formula I, i.e. compounds of the formula I in which R¹ and R⁹ are both phenoxy and the R^{a}, R^{b}, R², R³, R⁴, R⁵, R⁶, R⁷, R⁸, R¹⁰, R¹¹ and R¹² radicals are all hydrogen, or R⁵ and R⁸ are both methoxy and the R^{a}, R^{b}, R¹, R², R³, R⁴, R⁶, R⁷, R⁹, R¹⁰, R¹¹ and R¹² radicals are all hydrogen.

### DESCRIPTION OF FIGURES

FIG. 1 shows a solar cell which is suitable for the use of indanthrene compounds and has normal structure:

FIG. 2 shows a solar cell with inverse structure.

FIG. 3 shows the structure of a solar cell with normal structure and with a donor-acceptor interface in the form of a bulk heterojunction.

FIG. 4 shows the structure of a solar cell with inverse structure and with a donor-acceptor interface in the form of a bulk heterojunction.

FIG. 5 shows the structure of a tandem cell.

FIG. 6 shows the structure of a solar cell with a donor-acceptor interface in the form of a bulk heterojunction configured as a gradient.

FIG. 7 shows the absorption spectrum of a vapor-deposited film of indanthrene blue.

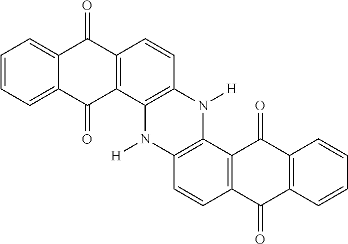

FIG. 8 shows the absorption spectrum of a vapor-deposited film of 4,4′-dimethoxyindanthrone.

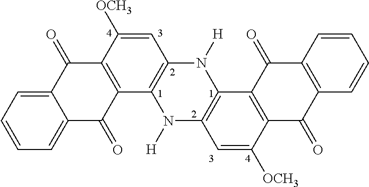

FIG. 9 shows the absorption spectrum of a vapor-deposited film of 5,5′-diphenoxyindanthrone.

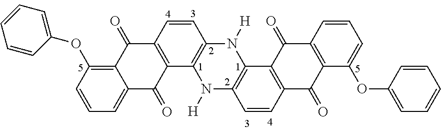

### DETAILED DESCRIPTION OF THE INVENTION

In the context of the invention, the expression “photoactive region” represents a photoactive heterojunction formed by at least one electron-conducting organic material and at least one hole-conducting organic material.

In the context of the present application, an organic material is referred to as “hole-conducting” when the charge carriers which are formed as a result of light absorption and charge separation at a heterojunction (“photogenerated charge carriers”) are transported within the material in the form of holes. Accordingly, an organic material is referred to as “electron-conducting” when photogenerated charge carriers are transported within the material in the form of electrons.

A “heterojunction” refers to an interface region between the electron-conducting and the hole-conducting material.

A “photoactive heterojunction” refers to a heterojunction between the electron-conducting and the hole-conducting material when excited states formed by light absorption in the electron-conducting and/or the hole-conducting material (“excitons”) which are separated in the region of the heterojunction into the individual charge carriers, namely electrons and holes, which are then in turn transported through the electron-conducting material/the hole-conducting material to electrical contacts, where electrical energy can be drawn off.

A “flat heterojunction” refers to a heterojunction between the electron-conducting and the hole-conducting material when the interface between the electron-conducting and the hole-conducting material is formed as an essentially cohesive surface between the two material regions, namely one region of the electron-conducting material and one region of the hole-conducting material (cf. C. W. Tang, Appl. Phys. Lett, 48 (2), 183-185 (1986) or N. Karl et al., Mol. Cryst. Liq. Cryst, 252, 243-258 (1994)).

A “bulk heterojunction” refers to a heterojunction between the electron-conducting and the hole-conducting material when the electron-conducting material and the hole-conducting material are at least partly mixed with one another, such that the interface between the electron-conducting and the hole-conducting material comprises a multitude of interface sections distributed over the volume of the material mixture (cf., for example, C. J. Brabec et al., Adv. Funct. Mater., 11(1), 15 (2001)).

Here and hereinafter, in relation to the indanthrene compound used in accordance with the invention, the terms indanthrene compound and indanthrone compound are used synonymously.

The indanthrene compounds used in accordance with the invention are photoactive materials having a high absorption coefficient in the long-wavelength range of the solar spectrum. They are especially suitable for use in a component cell of a tandem cell, in order to achieve a maximum light yield combined with a high voltage. It is thus possible to further improve the efficiency of organic solar cells.

In the context of the invention, the expression “unsubstituted or substituted alkyl, alkoxy, alkylthio, cycloalkyl, aryl, aryloxy, arylthio, hetaryl, hetaryloxy, hetarylthio, oligo(het)aryl, oligo(het)aryloxy and oligo(het)alkylthio” represents unsubstituted or substituted alkyl, unsubstituted or substituted alkoxy, unsubstituted or substituted alkylthio, unsubstituted or substituted cycloalkyl, unsubstituted or substituted aryl, unsubstituted or substituted aryloxy, unsubstituted or substituted arylthio, unsubstituted or substituted hetaryl, unsubstituted or substituted hetaryloxy, unsubstituted or substituted hetarylthio, unsubstituted or substituted oligo(het)aryl, unsubstituted or substituted oligo(het)aryloxy and unsubstituted or substituted oligo(het)arylthio.

In the context of the present invention, the expression “alkyl” comprises straight-chain or branched alkyl. Alkyl is preferably C₁-C₃₀-alkyl, especially C₁-C₂₀-alkyl and most preferably C₁-C₁₂-alkyl. Examples of alkyl groups are especially methyl, ethyl, n-propyl, isopropyl, n-butyl, isobutyl, sec-butyl, tert-butyl, n-pentyl, n-hexyl, n-heptyl, n-octyl, n-nonyl, n-decyl, n-undecyl, n-dodecyl, n-tridecyl, n-tetradecyl, n-hexadecyl, n-octadecyl and n-eicosyl.

The expression “alkyl” also comprises alkyl radicals whose carbon chains may be interrupted by one or more nonadjacent groups selected from —O—, —S—, —NR^{c}—, —C(═O)—, —S(═O)— and/or —S(═O)₂—. R^{c} is preferably hydrogen, alkyl, cycloalkyl, heterocycloalkyl, aryl or hetaryl.

Substituted alkyl groups may, depending on the length of the alkyl chain, have one or more (e.g. 1, 2, 3, 4, 5 or more than 5) substituents. These are preferably each independently selected from cycloalkyl, heterocycloalkyl, aryl, hetaryl, fluorine, chlorine, bromine, hydroxyl, mercapto, cyano, nitro, nitroso, formyl, acyl, COOH, carboxylate, alkylcarbonyloxy, carbamoyl, SO₃H, sulfonate, sulfamino, sulfamide, amidino, NE¹E² where E¹ and E² are each independently hydrogen, alkyl, cycloalkyl, heterocycloalkyl, aryl or hetaryl. Cycloalkyl, heterocycloalkyl, aryl and hetaryl substituents of the alkyl groups may in turn be unsubstituted or substituted; suitable substituents are the substituents mentioned below for these groups.

Carboxylate and sulfonate respectively represent a derivative of a carboxylic acid function and a sulfonic acid function, especially a metal carboxylate or sulfonate, a carboxylic ester or sulfonic ester function or a carboxamide or sulfonamide function.

Aryl-substituted alkyl radicals (“aralkyl”, also referred to hereinafter as arylalkyl) have at least one unsubstituted or substituted aryl group, as defined below. The alkyl group in “aralkyl” may bear at least one further substituent and/or be interrupted by one or more nonadjacent groups selected from —O—, —S—, —NR^{d}—, —C(═O)—, —S(═O)— and/or —S(═O)₂—. R^{d} is preferably hydrogen, alkyl, cycloalkyl, heterocycloalkyl, aryl or hetaryl. Aralkyl is preferably phenyl-C₁-C₁₀-alkyl, more preferably phenyl-C₁-C₄-alkyl, for example benzyl, 1-phenethyl, 2-phenethyl, 1-phenprop-1-yl, 2-phenprop-1-yl, 3-phenprop-1-yl, 1-phenbut-1-yl, 2-phenbut-1-yl, 3-phenbut-1-yl, 4-phenbut-1-yl, 1-phenbut-2-yl, 2-phenbut-2-yl, 3-phenbut-2-yl, 4-phenbut-2-yl, 1-(phenmeth)eth-1-yl, 1-(phenmethyl)-1-(methyl)eth-1-yl or 1-(phenmethyl)-1-(methyl)prop-1-yl; preferably benzyl and 2-phenethyl.

Halogen-substituted alkyl groups (“haloalkyl”) comprise a straight-chain or branched alkyl group in which at least one hydrogen atom or all hydrogen atoms are replaced by halogen. The halogen atoms are preferably selected from fluorine, chlorine and bromine, especially fluorine and chlorine. Examples of haloalkyl groups are especially chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, —CH₂—C₂F₅, —CF₂—C₂F₅, —CF(CF₃)₂, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl, nonafluorobutyl, 5-fluoro-1-pentyl, 5-chloro-1-pentyl, 5-bromo-1-pentyl, 5-iodo-1-pentyl, 5,5,5-trichloro-1-pentyl, undecafluoropentyl, 6-fluoro-1-hexyl, 6-chloro-1-hexyl, 6-bromo-1-hexyl, 6-iodo-1-hexyl, 6,6,6-trichloro-1-hexyl or dodecafluorohexyl.

The above remarks regarding unsubstituted or substituted alkyl also apply to unsubstituted or substituted alkoxy, unsubstituted or substituted alkylamino, unsubstituted or substituted alkylthio (alkylsulfanyl), etc.

In the context of the invention, “cycloalkyl” denotes a cycloaliphatic radical having preferably 3 to 10, more preferably 5 to 8, carbon atoms. Examples of cycloalkyl groups are especially cyclopropyl, cyclobutyl, cyclopentyl, cyclohexyl, cycloheptyl or cyclooctyl.

Substituted cycloalkyl groups may, depending on the ring size, have one or more (e.g. 1, 2, 3, 4, 5 or more than 5) substituents. These are preferably each independently selected from alkyl, alkoxy, alkylthio, cycloalkyl, heterocycloalkyl, aryl, hetaryl, fluorine, chlorine, bromine, hydroxyl, mercapto, cyano, nitro, nitroso, formyl, acyl, COOH, carboxylate, alkylcarbonyloxy, carbamoyl, SO₃H, sulfonate, sulfamino, sulfamide, amidino, NE³E⁴ where E³ and E⁴ are each independently hydrogen, alkyl, cycloalkyl, heterocycloalkyl, aryl or hetaryl. In the case of substitution, the cycloalkyl groups preferably bear one or more, for example one, two, three, four or five, C₁-C₆-alkyl groups. Examples of substituted cycloalkyl groups are especially 2- and 3-methyl-cyclopentyl, 2- and 3-ethylcyclopentyl, 2-, 3- and 4-methylcyclohexyl, 2-, 3- and 4-ethylcyclohexyl, 2-, 3- and 4-propylcyclohexyl, 2-, 3- and 4-isopropylcyclohexyl, 2-, 3- and 4-butylcyclohexyl, 2-, 3- and 4-sec.-butylcyclohexyl, 2-, 3- and 4-tert-butylcyclohexyl, 2-, 3- and 4-methylcycloheptyl, 2-, 3- and 4-ethylcycloheptyl, 2-, 3- and 4-propylcycloheptyl, 2-, 3- and 4-isopropylcycloheptyl, 2-, 3- and 4-butylcycloheptyl, 2-, 3- and 4-sec-butylcycloheptyl, 2-, 3- and 4-tert-butylcycloheptyl, 2-, 3-, 4- and 5-methylcyclooctyl, 2-, 3-, 4- and 5-ethylcyclooctyl, 2-, 3-, 4- and 5-propylcyclooctyl.

In the context of the present invention, the expression “aryl” comprises mono- or polycyclic aromatic hydrocarbon radicals having 6 to 18, preferably 6 to 14, more preferably 6 to 10, carbon atoms. Examples of aryl are especially phenyl, naphthyl, indenyl, fluorenyl, anthracenyl, phenanthrenyl, naphthacenyl, chrysenyl, pyrenyl, etc., and especially phenyl or naphthyl.

Substituted aryls may, depending on the number and size of their ring systems, have one or more (e.g. 1, 2, 3, 4, 5 or more than 5) substituents. These are preferably each independently selected from alkyl, alkoxy, alkylthio, cycloalkyl, heterocycloalkyl, aryl, hetaryl, fluorine, chlorine, bromine, hydroxyl, mercapto, cyano, nitro, nitroso, formyl, acyl, COOH, carboxylate, alkylcarbonyloxy, carbamoyl, SO₃H, sulfonate, sulfamino, sulfamide, amidino, NE⁵E⁶ where E⁵ and E⁶ are each independently hydrogen, alkyl, cycloalkyl, heterocycloalkyl, aryl or hetaryl. The alkyl, alkoxy, alkylamino, alkylthio, cycloalkyl, heterocycloalkyl, aryl and hetaryl substituents on the aryl may in turn be unsubstituted or substituted. Reference is made to the substituents mentioned above for these groups. The substituents on the aryl are preferably selected from alkyl, alkoxy, haloalkyl, haloalkoxy, aryl, fluorine, chlorine, bromine, cyano and nitro. Substituted aryl is more preferably substituted phenyl which generally bears 1, 2, 3, 4 or 5, preferably 1, 2 or 3, substituents.

Substituted aryl is preferably aryl substituted by at least one alkyl group (“alkaryl”, also referred to hereinafter as alkylaryl). Alkaryl groups may, depending on the size of the aromatic ring system, have one or more (e.g. 1, 2, 3, 4, 5, 6, 7, 8, 9 or more than 9) alkyl substituents. The alkyl substituents may be unsubstituted or substituted. In this regard, reference is made to the above statements regarding unsubstituted and substituted alkyl. In a preferred embodiment, the alkaryl groups have exclusively unsubstituted alkyl substituents. Alkaryl is preferably phenyl which bears 1, 2, 3, 4 or 5, preferably 1, 2 or 3, more preferably 1 or 2, alkyl substituents.

Aryl which bears one or more radicals is, for example, 2-, 3- and 4-methylphenyl, 2,4-, 2,5-, 3,5- and 2,6-dimethylphenyl, 2,4,6-trimethylphenyl, 2-, 3- and 4-ethylphenyl, 2,4-, 2,5-, 3,5- and 2,6-diethylphenyl, 2,4,6-triethylphenyl, 2-, 3- and 4-propylphenyl, 2,4-, 2,5-, 3,5- and 2,6-dipropylphenyl, 2,4,6-tripropylphenyl, 2-, 3- and 4-isopropylphenyl, 2,4-, 2,5-, 3,5- and 2,6-diisopropylphenyl, 2,4,6-triisopropylphenyl, 2-, 3- and 4-butylphenyl, 2,4-, 2,5-, 3,5- and 2,6-dibutylphenyl, 2,4,6-tributylphenyl, 2-, 3- and 4-isobutylphenyl, 2,4-, 2,5-, 3,5- and 2,6-diisobutylphenyl, 2,4,6-triisobutylphenyl, 2-, 3- and 4-sec-butylphenyl, 2,4-, 2,5-, 3,5- and 2,6-di-sec-butylphenyl, 2,4,6-tri-sec-butylphenyl, 2-, 3- and 4-tert-butylphenyl, 2,4-, 2,5-, 3,5- and 2,6-di-tert-butylphenyl and 2,4,6-tri-tert-butylphenyl; 2-, 3- and 4-methoxyphenyl, 2,4-, 2,5-, 3,5- and 2,6-dimethoxyphenyl, 2,4,6-trimethoxyphenyl, 2-, 3- and 4-ethoxyphenyl, 2,4-, 2,5-, 3,5- and 2,6-diethoxyphenyl, 2,4,6-triethoxyphenyl, 2-, 3- and 4-propoxyphenyl, 2,4-, 2,5-, 3,5- and 2,6-dipropoxyphenyl, 2-, 3- and 4-isopropoxyphenyl, 2,4-, 2,5-, 3,5- and 2,6-diisopropoxyphenyl and 2-, 3- and 4-butoxyphenyl; 2-, 3- and 4-cyanophenyl.

The above remarks regarding unsubstituted or substituted aryl also apply to unsubstituted or substituted aryloxy and unsubstituted or substituted arylthio. Examples of aryloxy are phenoxy, naphthyloxy or anthracenyloxy. Examples of arylthio are phenylthio, naphthylthio or anthracenylthio.

In the context of the present invention, the expression “heterocycloalkyl” comprises nonaromatic, unsaturated or fully saturated, cycloaliphatic groups having generally 5 to 8 ring atoms, preferably 5 or 6 ring atoms. In the heterocycloalkyl groups, compared to the corresponding cycloalkyl groups, 1, 2, 3, 4 or more than 4 of the ring carbon atoms are replaced by heteroatoms or heteroatom-containing groups. The heteroatoms or heteroatom-containing groups are preferably selected from —O—, —S—, —NR^{e}—, —C(═O)—, —S(═O)— and/or —S(═O)₂—. R^{e} is preferably hydrogen, alkyl, cycloalkyl, heterocycloalkyl, aryl or hetaryl. Heterocycloalkyl is unsubstituted or optionally bears one or more, e.g. 1, 2, 3, 4, 5, 6 or 7, identical or different radicals. These are preferably each independently selected from alkyl, alkoxy, alkylamino, alkylthio, cycloalkyl, heterocycloalkyl, aryl, hetaryl, fluorine, chlorine, bromine, hydroxyl, mercapto, cyano, nitro, nitroso, formyl, acyl, COON, carboxylate, alkylcarbonyloxy, carbamoyl, SO₃H, sulfonate, sulfamino, sulfamide, amidino, NE⁵E⁶ where E⁵ and E⁶ are each independently hydrogen, alkyl, cycloalkyl, heterocycloalkyl, aryl or hetaryl. Examples of heterocycloalkyl groups are especially pyrrolidinyl, piperidinyl, 2,2,6,6-tetramethylpiperidinyl, imidazolidinyl, pyrazolidinyl, oxazolidinyl, morpholidinyl, thiazolidinyl, isothiazolidinyl, isoxazolidinyl, piperazinyl, tetrahydrothiophenyl, dihydrothien-2-yl, tetrahydrofuranyl, dihydrofuran-2-yl, tetrahydropyranyl, 1,2-oxazolin-5-yl, 1,3-oxazolin-2-yl and dioxanyl.

Substituted heterocycloalkyl groups may, depending on the ring size, have one or more (e.g. 1, 2, 3, 4, 5 or more than 5) substituents. These are preferably each independently selected from alkyl, alkoxy, alkylthio, cycloalkyl, heterocycloalkyl, aryl, hetaryl, fluorine, chlorine, bromine, hydroxyl, mercapto, cyano, nitro, nitroso, formyl, acyl, COOH, carboxylate, alkylcarbonyloxy, carbamoyl, SO₃H, sulfonate, sulfamino, sulfamide, amidino, NE⁷E⁸ where E⁷ and E⁸ are each independently hydrogen, alkyl, cycloalkyl, heterocycloalkyl, aryl or hetaryl. In the case of substitution, the heterocycloalkyl groups preferably bear one or more, for example one, two, three, four or five, C₁-C₆-alkyl groups.

In the context of the present invention, the expression “heteroaryl” (hetaryl) comprises heteroaromatic, mono- or polycyclic groups. In addition to the ring carbon atoms, these have 1, 2, 3, 4 or more than 4 of the ring heteroatoms. The heteroatoms are preferably selected from oxygen, nitrogen, selenium and sulfur. The hetaryl groups have preferably 5 to 18, e.g. 5, 6, 8, 9, 10, 11, 12, 13 or 14, ring atoms.

Monocyclic hetaryl groups are preferably 5- or 6-membered hetaryl groups, such as 2-furyl (furan-2-yl), 3-furyl (furan-3-yl), 2-thienyl (thiophen-2-yl), 3-thienyl (thiophen-3-yl), selenophen-2-yl, selenophen-3-yl, 1 H-pyrrol-2-yl, 1 H-pyrrol-3-yl, pyrrol-1-yl, imidazol-2-yl, imidazol-1-yl, imidazol-4-yl, pyrazol-1-yl, pyrazol-3-yl, pyrazol-4-yl, pyrazol-5-yl, 3-isoxazolyl, 4-isoxazolyl, 5-isoxazolyl, 3-isothiazolyl, 4-isothiazolyl, 5-isothiazolyl, 2-oxazolyl, 4-oxazolyl, 5-oxazolyl, 2-thiazolyl, 4-thiazolyl, 5-thiazolyl, 1,2,4-oxadiazol-3-yl, 1,2,4-oxadiazol-5-yl, 1,3,4-oxadiazol-2-yl, 1,2,4-thiadiazol-3-yl, 1,2,4-thiadiazol-5-yl, 1,3,4-thiadiazol-2-yl, 4H-[1,2,4]-triazol-3-yl, 1,3,4-triazol-2-yl, 1,2,3-triazol-1-yl, 1,2,4-triazol-1-yl, pyridin-2-yl, pyridin-3-yl, pyridin-4-yl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

Polycyclic hetaryl groups have 2, 3, 4 or more than 4 fused rings. The fused-on rings may be aromatic, saturated or partly unsaturated. Examples of polycyclic hetaryl groups are quinolinyl, isoquinolinyl, indolyl, isoindolyl, indolizinyl, benzofuranyl, isobenzofuranyl, benzothiophenyl, benzoxazolyl, benzisoxazolyl, benzthiazolyl, benzoxadiazolyl; benzothiadiazolyl, benzoxazinyl, benzopyrazolyl, benzimidazolyl, benzotriazolyl, benzotriazinyl, benzoselenophenyl, thienothiophenyl, thienopyrimidyl, thiazolothiazolyl, dibenzopyrrolyl (carbazolyl), dibenzofuranyl, dibenzothiophenyl, naphtho[2,3-b]thiophenyl, naphtha[2,3-b]furyl, dihydroindolyl, dihydroindolizinyl, dihydroisoindolyl, dihydroquinolinyl, dihydroisoquinolinyl.

Substituted hetaryl groups may, depending on the number and size of their ring systems, have one or more (e.g. 1, 2, 3, 4, 5 or more than 5) substituents. These are preferably each independently selected from alkyl, alkoxy, alkylthio, cycloalkyl, heterocycloalkyl, aryl, hetaryl, fluorine, chlorine, bromine, hydroxyl, mercapto, cyano, nitro, nitroso, formyl, acyl, COOH, carboxylate, alkylcarbonyloxy, carbamoyl, SO₃H, sulfonate, sulfamino, sulfamide, amidino, NE⁹E¹⁹ where E⁹ and E¹⁰ are each independently hydrogen, alkyl, cycloalkyl, heterocycloalkyl, aryl or hetaryl. Halogen substituents are preferably fluorine, chlorine or bromine. The substituents are preferably selected from C₁-C₆-alkyl, C₁-C₆-alkoxy, hydroxyl, carboxyl, halogen and cyano.

The above remarks regarding unsubstituted or substituted hetaryl also apply to unsubstituted or substituted hetaryloxy and unsubstituted or substituted hetarylthio.

In the context of the present application, the expression “oligo(het)aryl” denotes unsubstituted or substituted groups having at least two repeat units. The repeat units may all have the same definition, some of the repeat units may have different definitions or all repeat units may have different definitions. The repeat unit is selected from aryldiyl groups, hetaryldiyl groups and combinations thereof. The aryldiyl group is a divalent group derived from an aromatic, preferably a group derived from benzene or naphthalene, such as 1,2-phenylene (o-phenylene), 1,3-phenylene (m-phenylene), 1,4-phenylene (p-phenylene), 1,2-naphthylene, 2,3-naphthylene, 1,4-naphthylene, etc. The hetaryldiyl group is a divalent group derived from a heteroaromatic, preferably a group derived from thiophene or furan. The terminal group of the oligo(het)aryl groups is a monovalent group. This preferably likewise derives from the aforementioned repeat units. The oligo(het)aryl groups may be unsubstituted or substituted. Substituted oligo(het)aryls may, depending on the number and size of their ring systems, have one or more (e.g. 1, 2, 3, 4, 5, 6, 7, 8, 9 or more than 9) substituents. These substituents are preferably each independently selected from unsubstituted alkyl, haloalkyl, fluorine or chlorine.

Suitable repeat units are as follows:

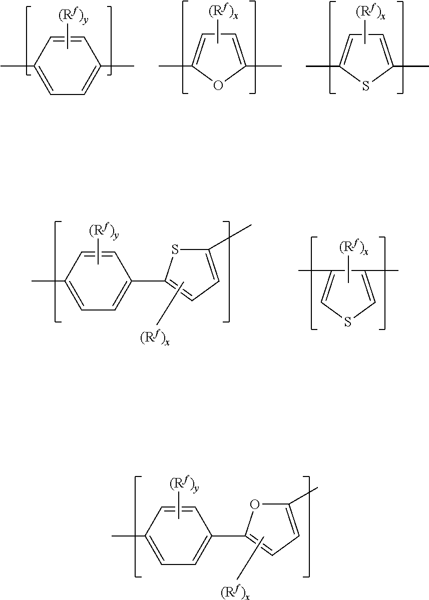

in which the R¹ radicals are each independently alkyl, alkoxy, haloalkyl, fluorine or chlorine, y is 0, 1, 2, 3 or 4 and x is 0, 1 or 2.

Preferred oligoaryl groups are biphenylyl, p-terphenylyl, m-terphenylyl, o-terphenylyl, quaterphenylyl, e.g. p-quaterphenylyl, quinquephenylyl, e.g. p-quinquephenylyl.

Preferred oligohetaryl groups are:

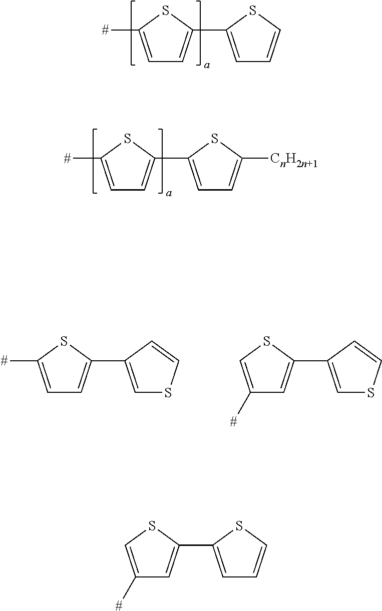

in which # represents a bonding site to the rest of the molecule, and

a is 1, 2, 3, 4, 5, 6, 7 or 8,

n is 1 to 12, preferably 1 to 6.

a is preferably 1 or 2.

The CₙH₂ₙ₊₁ radical is preferably methyl, ethyl, n-propyl, isopropyl, n-butyl, sec-butyl, tert-butyl, n-pentyl or n-hexyl.

Examples of oligohetaryl groups are 2,2 -bithiophen-5-yl and 5″-hexyl-2,2″-bithiophen-5-yl.

Halogen represents fluorine, chlorine, bromine or iodine. Halogen preferably represents fluorine or chlorine.

Specific examples of the R¹ to R¹² radicals and, in accordance with the above definition, also R^{a} and R^{b}, specified in the above formula (I) and the formulae which follow are:

methyl, ethyl, n-propyl, isopropyl, n-butyl, isobutyl, sec-butyl, tert-butyl, n-pentyl, n-hexyl, n-heptyl, n-octyl, n-nonyl, n-decyl, n-undecyl, n-dodecyl, n-tridecyl, n-tetradecyl, n-hexadecyl, n-octadecyl and n-eicosyl, 2-methoxyethyl, 2-ethoxyethyl, 2-propoxyethyl, 2-butoxyethyl, 3-methoxypropyl, 3-ethoxypropyl, 3-propoxypropyl, 3-butoxypropyl, 4-methoxybutyl, 4-ethoxybutyl, 4-propoxybutyl, 3,6-dioxaheptyl, 3,6-dioxaoctyl, 4,8-dioxanonyl, 3,7-dioxaoctyl, 3,7-dioxanonyl, 4,7-dioxaoctyl, 4,7-dioxanonyl, 2- and 4-butoxybutyl, 4,8-dioxadecyl, 3,6,9-trioxadecyl, 3,6,9-trioxaundecyl, 3,6,9-trioxadodecyl, 3,6,9,12-tetraoxatridecyl and 3,6,9,12-tetraoxatetradecyl;

2-methylthioethyl, 2-ethylthioethyl, 2-propylthioethyl, 2-butylthioethyl, 3-methylthiopropyl, 3-ethylthiopropyl, 3-propylthiopropyl, 3-butylthiopropyl, 4-methylthiobutyl, 4-ethylthiobutyl, 4-propylthiobutyl, 3,6-dithiaheptyl, 3,6-dithiaoctyl, 4,8-dithianonyl, 3,7-dithiaoctyl, 3,7-dithianonyl, 2- and 4-butylthiobutyl, 4,8-dithiadecyl, 3,6,9-trithiadecyl, 3,6,9-trithiaundecyl, 3,6,9-trithiadodecyl, 3,6,9,12-tetrathiatridecyl and 3,6,9,12-tetrathiatetradecyl;

2-monomethyl- and 2-monoethylaminoethyl, 2-dimethylaminoethyl, 2- and 3-dimethylaminopropyl, 3-monoisopropylaminopropyl, 2- and 4-monopropylaminobutyl, 2- and 4-dimethylaminobutyl, 6-methyl-3,6-diazaheptyl, 3,6-dimethyl-3,6-diazaheptyl, 3,6-diazaoctyl, 3,6-dimethyl-3,6-diazaoctyl, 9-methyl-3,6,9-triazadecyl, 3,6,9-trimethyl-3,6,9-triazadecyl, 3,6,9-triazaundecyl, 3,6,9-trimethyl-3,6,9-triazaundecyl, 12-methyl-3,6,9,12-tetraazatridecyl and 3,6,9,12-tetramethyl-3,6,9,12-tetraazatridecyl;

(1-ethylethylidene)aminoethylene, (1-ethylethylidene)aminopropylene, (1-ethylethylidene)aminobutylene, (1-ethylethylidene)aminodecylene and (1-ethylethylidene)aminododecylene;

propan-2-on-1-yl, butan-3-on-1-yl, butan-3-on-2-yl and 2-ethylpentan-3-on-1-yl;

2-methylsulfinylethyl, 2-ethylsulfinylethyl, 2-propylsulfinylethyl, 2-isopropylsulfinylethyl, 2-butylsulfinylethyl, 2- and 3-methylsulfinylpropyl, 2- and 3-ethylsulfinylpropyl, 2- and 3-propylsulfinylpropyl, 2- and 3-butylsulfinylpropyl, 2- and 4-methylsulfinylbutyl, 2- and 4-ethylsulfinylbutyl, 2- and 4-propylsulfinylbutyl and 4-butylsulfinylbutyl;

2-methylsulfonylethyl, 2-ethylsulfonylethyl, 2-propylsulfonylethyl, 2-isopropylsulfonylethyl, 2-butylsulfonylethyl, 2- and 3-methylsulfonylpropyl, 2- and 3-ethylsulfonylpropyl, 2- and 3-propylsulfonylpropyl, 2- and 3-butylsulfonylproypl, 2- and 4-methylsulfonylbutyl, 2- and 4-ethylsulfonylbutyl, 2- and 4-propylsulfonylbutyl and 4-butylsulfonylbutyl;

carboxymethyl, 2-carboxyethyl, 3-carboxypropyl, 4-carboxybutyl, 5-carboxypentyl, 6-carboxyhexyl, 8-carboxyoctyl, 10-carboxydecyl, 12-carboxydodecyl and 14-carboxyl-tetradecyl;

sulfomethyl, 2-sulfoethyl, 3-sulfopropyl, 4-sulfobutyl, 5-sulfopentyl, 6-sulfohexyl, 8-sulfooctyl, 10-sulfodecyl, 12-sulfododecyl and 14-sulfotetradecyl;

2-hydroxyethyl, 2- and 3-hydroxypropyl, 3- and 4-hydroxybutyl and 8-hydroxyl-4-oxaoctyl;

2-cyanoethyl, 3-cyanopropyl, 3- and 4-cyanobutyl;

2-chloroethyl, 2- and 3-chloropropyl, 2-, 3- and 4-chlorobutyl, 2-bromoethyl, 2- and 3-bromopropyl and 2-, 3- and 4-bromobutyl;

2-nitroethyl, 2- and 3-nitropropyl and 2-, 3- and 4-nitrobutyl;

methoxy, ethoxy, propoxy, butoxy, pentoxy and hexoxy;

methylthio, ethylthio, propylthio, butylthio, pentylthio and hexylthio;

methylamino, ethylamino, propylamino, butylamino, pentylamino, hexylamino, dicyclopentylamino, dicyclohexylamino, dicycloheptylamino, diphenylamino and dibenzylamino;

formylamino, acetylamino, propionylamino and benzoylamino;

carbamoyl, methylaminocarbonyl, ethylaminocarbonyl, propylaminocarbonyl, butyl-aminocarbonyl, pentylaminocarbonyl, hexylaminocarbonyl, heptylaminocarbonyl, octylaminocarbonyl, nonylaminocarbonyl, decylaminocarbonyl and phenylamino-carbonyl;

aminosulfonyl, n-dodecylaminosulfonyl, N,N-diphenylaminosulfonyl, and N,N-bis(4-chlorophenyl)aminosulfonyl;

methoxycarbonyl, ethoxycarbonyl, propoxycarbonyl hexoxycarbonyl, dodecyloxycarbonyl, octadecyloxycarbonyl, phenoxycarbonyl, (4-tert-butylphenoxy)carbonyl and (4-chlorophenoxy)carbonyl;

methoxysulfonyl, ethoxysulfonyl, propoxysulfonyl, butoxysulfonyl, hexoxysulfonyl, dodecyloxysulfonyl, octadecyloxysulfonyl, phenoxysulfonyl, 1- and 2-naphthyloxysulfonyl, (4-tert-butylphenoxy)sulfonyl and (4-chlorophenoxy)sulfonyl;

diphenylphosphino, di(o-tolyl)phosphino and diphenyiphosphinoxido;

fluorine, chlorine, bromoine and iodine;

cyclopropyl, cyclobutyl, cyclopentyl, 2- and 3-methylcyclopentyl, 2- and 3-ethylcyclopentyl, cyclohexyl, 2-, 3- and 4-methylcyclohexyl, 2-, 3- and 4-ethylcyclohexyl, 3- and 4-propylcyclohexyl, 3- and 4-isopropylcyclohexyl, 3- and 4-butylcyclohexyl, 3- and 4-sec-butylcyclohexyl, 3- and 4-tert-butylcyclohexyl, cycloheptyl, 2-, 3- and 4-methyl-cycloheptyl, 2-, 3- and 4-ethylcycloheptyl, 3- and 4-propylcycloheptyl, 3- and 4-iso-propylcycloheptyl, 3- and 4-butylcycloheptyl, 3- and 4-sec-butylcycloheptyl, 3- and 4-tert-butylcycloheptyl, cyclooctyl, 2-, 3-, 4- and 5-methylcyclooctyl, 2-, 3-, 4- and 5-ethylcyclooctyl and 3-, 4- and 5-propylcyclooctyl; 3- and 4-hydroxycyclohexyl, 3- and 4-nitrocyclohexyl and 3- and 4-chlorocyclohexyl;

1-, 2- and 3-cyclopentenyl, 1-, 2-, 3- and 4-cyclohexenyl, 1-, 2- and 3-cycloheptenyl and 1-, 2-, 3- and 4-cyclooctenyl;

2-dioxanyl, 4-morpholinyl, 4-thiomorpholinyl, 2- and 3-tetrahydrofuryl, 1-, 2- and 3-pyrrolidinyl, 1-piperazyl, 1-diketopiperazyl and 1-, 2-, 3- and 4-piperidyl;

phenyl, 2-naphthyl, 2- and 3-pyrryl, 2-, 3- and 4-pyridyl, 2-, 4- and 5-pyrimidyl, 3-, 4- and 5-pyrazolyl, 2-, 4- and 5-imidazolyl, 2-, 4- and 5-thiazolyl, 3-(1,2,4-triazyl), 2-(1,3,5-triazyl), quinaldin-6-yl, 3-, 5-, 6- and 8-quinolinyl, 2-benzoxazolyl, 2-benzothiazolyl, 5-benzothiadiazolyl, 2- and 5-benzimidazolyl and 1- and 5-isoquinolyl;

1-, 2-, 3-, 4-, 5-, 6- and 7-indolyl, 1-, 2-, 3-, 4-, 5-, 6- and 7-isoindolyl, 5-(4-methylisoindolyl), 5-(4-phenylisoindolyl), 1-, 2-, 4-, 6-, 7- and 8-(1,2,3,4-tetrahydroisoquinolinyl), 3-(5-phenyl)-(1,2,3,4-tetrahydroisoquinolinyl), 5-(3-dodecyl-(1,2,3,4-tetrahydroisoquinolinyl), 1-, 2-, 3-, 4-, 5-, 6-, 7- and 8-(1,2,3,4-tetrahydroquinolinyl) and 2-, 3-, 4-, 5-, 6-, 7- and 8-chromanyl, 2-, 4- and 7-quinolinyl, 2-(4-phenylquinolinyl) and 2-(5-ethylquinolinyl);

2-, 3- and 4-methylphenyl, 2,4-, 3,5- and 2,6-dimethylphenyl, 2,4,6-trimethylphenyl, 2-, 3- and 4-ethylphenyl, 2,4-, 3,5- and 2,6-diethylphenyl, 2,4,6-triethylphenyl, 2-, 3- and 4-propylphenyl, 2,4-, 3,5- and 2,6-dipropylphenyl, 2,4,6-tripropylphenyl, 2-, 3- and 4-isopropylphenyl, 2,4-, 3,5- and 2,6-diisopropylphenyl, 2,4,6-triisopropylphenyl, 2-, 3- and 4-butylphenyl, 2,4-, 3,5- and 2,6-dibutylphenyl, 2,4,6-tributylphenyl, 2-, 3- and 4-isobutylphenyl, 2,4-, 3,5- and 2,6-diisobutylphenyl, 2,4,6-triisobutylphenyl, 2-, 3- and 4-sec-butylphenyl, 2,4-, 3,5- and 2,6-di-sec-butylphenyl and 2,4,6-tri-sec-butylphenyl; 2-, 3- and 4-methoxyphenyl, 2,4-, 3,5- and 2,6-dimethoxyphenyl, 2,4,6-trimethoxyphenyl, 2-, 3- and 4-ethoxyphenyl, 2,4-, 3,5- and 2,6-diethoxyphenyl, 2,4,6-triethoxyphenyl, 2-, 3- and 4-propoxyphenyl, 2,4-, 3,5- and 2,6-dipropoxyphenyl, 2-, 3- and 4-isopropoxyphenyl, 2,4- and 2,6-diisopropoxyphenyl and 2-, 3- and 4-butoxyphenyl; 2-, 3- and 4-chlorophenyl and 2,4-, 3,5- and 2,6-dichlorophenyl; 2-, 3- and 4-hydroxyphenyl and 2,4-, 3,5- and 2,6-dihydroxyphenyl; 2-, 3- and 4-cyanophenyl; 3- and 4-carboxyphenyl; 3- and 4-carboxamidophenyl, 3- and 4-N-methylcarboxamido-phenyl and 3- and 4-N-ethylcarboxamidophenyl; 3- and 4-acetylaminophenyl, 3- and 4-propionylaminophenyl and 3- and 4-butyrylaminophenyl; 3- and 4-N-phenylamino-phenyl, 3- and 4-N-(o-tolyl)aminophenyl, 3- and 4-N-(m-tolyl)aminophenyl and 3- and 4-(p-tolyl)aminophenyl; 3- and 4-(2-pyridyl)aminophenyl, 3- and 4-(3-pyridyl)aminophenyl, 3- and 4-(4-pyridyl)aminophenyl, 3- and 4-(2-pyrimidyl)aminophenyl and 4-(4-pyrimidyl)aminophenyl;

4-phenylazophenyl, 4-(1-naphthylazo)phenyl, 4-(2-naphthylazo)phenyl, 4-(4-naphthylazo)phenyl, 4-(2-pyriylazo)phenyl, 4-(3-pyridylazo)phenyl, 4-(4-pyridylazo)phenyl, 4-(2-pyrimidylazo)phenyl, 4-(4-pyrimidylazo)phenyl and 4-(5-pyrimidylazo)phenyl;

phenoxy, phenylthio, 2-naphthoxy, 2-naphthylthio, 2-, 3- and 4-pyridyloxy, 2-, 3- and 4-pyridylthio, 2-, 4- and 5-pyrimidyloxy and 2-, 4- and 5-pyrimidylthio.

Preferred fluorinated R^{a}, R^{b} and R¹ to R¹² radicals are as follows:

2,2,2-trifluoroethyl, 2,2,3,3,3-pentafluoropropyl, 2,2-difluoroethyl, 2,2,3,3,4,4,4-heptafluorobutyl, 2,2,3,3,3-pentafluoropropyl, 1H,1H-pentadecafluorooctyl, 3-bromo-3,3-difluoropropyl, 3,3,3-trifluoropropyl, 3,3,3-trifluoropropyl, 1H,1H,2H,2H-perfluorodecyl, 3-(perfluorooctyl)propyl, 4,4-difluorobutyl, 4,4,4-trifluorobutyl, 5,5,6,6,6-pentafluorohexyl, 2,2-difluoropropyl, 2,2,2-trifluoro-1-phenylethylamino, 1-benzyl-2,2,2-trifluoroethyl, 2-bromo-2,2-difluoroethyl, 2,2,2-trifluoro-1-pyridin-2-ylethyl, 2,2-difluoropropyl, 2,2,2-trifluoro-1-(4-methoxyphenyl)ethylamino, 2,2,2-trifluoro-1-phenylethyl, 2,2-difluoro-1-phenylethyl, 1-(4-bromophenyl)-2,2,2-trifluoroethyl, 3-bromo-3,3-difluoropropyl, 3,3,3-trifluoropropylamino, 3,3,3-trifluoro-n-propyl, 1H,1H,2H,2H-perfluorodecyl, 3-(perfluorooctyl)propyl, pentafluorophenyl, 2,3,5,6-tetrafluorophenyl, 4-cyano(2,3,5,6)-tetrafluorophenyl, 4-carboxyl-2,3,5,6-tetrafluorophenyl, 2,4-difluorophenyl, 2,4,5-trifluorophenyl, 2,4,6-trifluorophenyl, 2,5-difluorophenyl, 2-fluoro-5-nitrophenyl, 2-fluoro-5-trifluoromethylphenyl, 2-fluoro-5-methylphenyl, 2,6-difluorophenyl, 4-carboxamido-2,3,5,6-tetrafluorophenyl, 2-bromo-4,6-difluorophenyl, 4-bromo-2-fluorophenyl, 2,3-difluorophenyl, 4-chloro-2-fluorophenyl, 2,3,4-trifluorophenyl, 2-fluoro-4-iodophenyl, 4-bromo-2,3,5,6-tetrafluorophenyl, 2,3,6-trifluorophenyl, 2-bromo-3,4,6-trifluorophenyl, 2-bromo-4,5,6-trifluorophenyl, 4-bromo-2,6-difluorophenyl, 2,3,4,5-tetrafluorophenyl, 2,4-difluoro-6-nitrophenyl, 2-fluoro-4-nitrophenyl, 2-chloro-6-fluorophenyl, 2-fluoro-4-methylphenyl, 3-chloro-2,4-difluorophenyl, 2,4-dibromo-6-fluorophenyl, 3,5-dichloro-2,4-difluorophenyl, 4-cyano-1-fluorophenyl, 1-chloro-4-fluorophenyl, 2-fluoro-3-trifluoromethylphenyl, 2-trifluoromethyl-6-fluorophenyl, 2,3,4,6-tetrafluorophenyl, 3-chloro-2-fluorophenyl, 5-chloro-2-fluorophenyl, 2-bromo-4-chloro-6-fluorophenyl, 2,3-dicyano-4,5,6-trifluorophenyl, 2,4,5-trifluoro-3-carboxyphenyl, 2,3,4-trifluoro-6-carboxyphenyl, 2,3,5-trifluorophenyl, 4-trifluoromethyl-2,3,5,6-tetrafluorophenyl, 1-fluoro-5-carboxyphenyl, 2-chloro-4,6-difluorophenyl, 6-bromo-3-chloro-2,4-difluorophenyl, 2,3,4-trifluoro-6-nitrophenyl, 2,5-difluoro-4-cyanophenyl, 2,5-difluoro-4-trifluoromethylphenyl, 2,3-difluoro-6-nitrophenyl, 4-trifluoromethyl-2,3-difluorophenyl, 2-bromo-4,6-difluorophenyl, 4-bromo-2-fluorophenyl, 2-nitrotetrafluorophenyl, 2,2′,3,3′,4′,5,5′,6,6′-nonafluorobiphenyl, 2-nitro-3,5,6-trifluorophenyl, 2-bromo-6-fluorophenyl, 4-chloro-2-fluoro-6-iodophenyl, 2-fluoro-6-carboxyphenyl, 2,4-difluoro-3-trifluorophenyl, 2-fluoro-4-trifluorophenyl, 2-fluoro-4-carboxyphenyl, 4-bromo-2,5-difluorophenyl, 2,5-dibromo-3,4,6-trifluorophenyl, 2-fluoro-5-methylsulfonylphenyl, 5-bromo-2-fluorophenyl, 2-fluoro-4-hydroxymethylphenyl, 3-fluoro-4-bromomethylphenyl, 2-nitro-4-trifluoromethylphenyl, 4-trifluoromethylphenyl, 2-bromo-4-trifluoromethylphenyl, 2-bromo-6-chloro-4-(trifluoromethyl)phenyl, 2-chloro-4-trifluoromethylphenyl, 3-nitro-4-(trifluoromethyl)phenyl, 2,6-dichloro-4-(trifluoromethyl)phenyl, 4-trifluorophenyl, 2,6-dibromo-4-(trifluoromethyl)phenyl, 4-trifluoromethyl-2,3,5,6-tetrafluorophenyl, 3-fluoro-4-trifluoromethylphenyl, 2,5-difluoro-4-trifluoromethylphenyl, 3,5-difluoro-4-trifluoromethylphenyl, 2,3-difluoro-4-trifluoromethylphenyl, 2,4-bis(trifluoromethyl)phenyl, 3-chloro-4-trifluoromethylphenyl, 2-bromo-4,5-di(trifluoromethyl)phenyl, 5-chloro-2-nitro-4-(trifluoromethyl)phenyl, 2,4,6-tris(trifluoromethyl)phenyl, 3,4-bis(trifluoromethyl)phenyl, 2-fluoro-3-trifluoromethylphenyl, 2-iodo-4-trifluoromethylphenyl, 2-nitro-4,5-bis(trifluoromethyl)phenyl, 2-methyl-4-(trifluoromethyl)phenyl, 3,5-dichloro-4-(trifluoromethyl)phenyl, 2,3,6-trichloro-4-(trifluoromethyl)phenyl, 4-(trifluoromethyl)benzyl, 2-fluoro-4-(trifluoromethyl)benzyl, 3-fluoro-4-(trifluoromethyl)benzyl, 3-chloro-4-(trifluoromethyl)benzyl, 4-fluorophenethyl, 3-(trifluoromethyl)phenethyl, 2-chloro-6-fluorophenethyl, 2,6-dichlorophenethyl, 3-fluorophenethyl, 2-fluorophenethyl, (2-trifluoromethyl)phenethyl, 4-fluorophenethyl, 3-fluorophenethyl, 4-trifluoromethylphenethyl, 2,3-difluorophenethyl, 3,4-difluorophenethyl, 2,4-difluorophenethyl, 2,5-difluorophenethyl, 3,5-difluorophenethyl, 2,6-difluorophenethyl, 4-(4-fluorophenyl)phenethyl, 3,5-di(trifluoromethyl)phenethyl, pentafluorophenethyl, 2,4-di(trifluoromethyl)phenethyl, 2-nitro-4-(trifluoromethyl)phenethyl, (2-fluoro-3-trifluoromethyl)phenethyl, (2-fluoro-5-trifluoromethyl)phenethyl, (3-fluoro-5-trifluoromethyl)phenethyl, (4-fluoro-2-trifluoromethyl)phenethyl, (4-fluoro-3-trifluoromethyl)phenethyl, (2-fluoro-6-trifluoromethyl)phenethyl, (2,3,6-trifluoro)phenethyl, (2,4,5-trifluoro)phenethyl, (2,4,6-trifluoro)phenethyl, (2,3,4-trifluoro)phenethyl, (3,4,5-trifluoro)phenethyl, (2,3,5-trifluoro)phenethyl, (2-chloro-5-fluoro)phenethyl, (3-fluoro-4-trifluoromethyl)phenethyl, (2-chloro-5-trifluoromethyl)phenethyl, (2-fluoro-3-chloro-5-trifluoromethyl)phenethyl, (2-fluoro-3-chloro)phenethyl, (4-fluoro-3-chloro)phenethyl, (2-fluoro-4-chloro)phenethyl, (2,3-difluoro-4-methyl)phenethyl, 2,6-difluoro-3-chlorophenethyl, (2,6-difluoro-3-methyl)phenethyl, (2-trifluoromethyl-5-chloro)phenethyl, (6-chloro-2-fluoro-5-methyl)phenethyl, (2,4-dichloro-5-fluoro)phenethyl, 5-chloro-2-fluorophenethyl, (2,5-difluoro-6-chloro)phenethyl, (2,3,4,5-tetrafluoro)phenethyl, (2-fluoro-4-trifluoromethyl)phenethyl, 2,3-(difluoro-4-trifluoromethyl)phenethyl, (2,5-di(trifluoromethyl))phenethyl, 2-fluoro-3,5-dibromophenethyl, (3-fluoro-4-nitro)phenethyl, (2-bromo-4-trifluoromethyl)phenethyl, 2-(bromo-5-fluoro)phenethyl, (2,6-difluoro-4-bromo)phenethyl, (2,6-difluoro-4-chloro)phenethyl, (3-chloro-5-fluoro)phenethyl, (2-bromo-5-trifluoromethyl)phenethyl and the like.

Specific examples of the R^{a}, R^{b} and R¹ to R¹² radicals specified in the above formula (I) and the formulae which follow are additionally: 1H,1H-perfluoro-C₂-C₃₀-alkyl or 1H,1H,2H,2H-perfluoro-C₃-C₃₀-alkyl, preferably 1H,1H-perfluoro-C₂-C₂₀-alkyl or 1H,1H,2H,2H-perfluoro-C₃-C₂₀-alkyl, especially 1H,1H-perfluoro-C₂-C₁₀-alkyl or 1H,1H,2H,2H-perfluoro-C₃-C₁₀-alkyl, such as 2,2,2-trifluoroethyl, 2,2,3,3,3-pentafluoropropyl, 2,2,3,3,4,4,4-heptafluorobutyl, 1H,1H-perfluoropentyl, 1H,1H-perfluorohexyl, 1H,1H-perfluoroheptyl, 1H,1H-pentadecafluorooctyl, 1 H,1 H-perfluorononyl, 1 H,1 H-perfluorodecyl, 3,3,3-trifluoropropyl, 3,3,4,4,4-pentafluorobutyl, 1H,1H,2H,2H-perfluoropentyl, 1H,1H,2H,2H-perfluorohexyl, 1H,1H,2H,2H-perfluoroheptyl, 1H,1H,2H,2H-perfluorooctyl, 1H,1H,2H,2H-perfluorononyl.

In the compounds of the general formula (I), the R^{a} and R^{b} radicals are preferably each independently selected from hydrogen, deuterium, unsubstituted alkyl, aralkyl, cycloalkyl, unsubstituted aryl and alkaryl.

In the compounds of the general formula (I), the R^{a} and R^{b} radicals are more preferably each independently selected from hydrogen, deuterium, C₁-C₁₂-alkyl, C₇-C₂₂-aralkyl, C₄-C₇-cycloalkyl, C₆-C₁₀-aryl and C₇-C₂₂-alkaryl.

In the compounds of the general formula (I), the R^{a} and R^{b} radicals preferably have the same definition.

In a specific embodiment, in the compounds of the general formula (I), the R^{a} and R^{b} radicals are both hydrogen.

In a further specific embodiment, in the compounds of the general formula (I), the R^{a} and R^{b} radicals are both deuterium.

In a further specific embodiment, in the compounds of the general formula (I), the R^{a} and R^{b} radicals are both C₁-C₆-alkyl, more specifically both methyl, ethyl, n-propyl, isopropyl, n-butyl, sec-butyl, tert-butyl, n-pentyl or n-hexyl.

In a further specific embodiment, in the compounds of the general formula (I), the R^{a} and R^{b} radicals are both phenyl, or both C₁-C₁₂-alkylphenyl, especially C₁-C₆-alkylphenyl, or both naphthyl.

Preferably, in the compounds of the general formula (I), the R¹ to R¹² radicals are each independently selected from hydrogen, F, Cl, hydroxyl, C₁-C₁₈-alkyl, C₁-C₁₂-alkoxy, C₁-C₆-alkylthio, C₇-C₂₂-aralkyl, C₇-C₂₂-aralkyloxy, C₇-C₂₂-aralkylthio, C₄-C₇-cycloalkyl, C₆-C₁₀-aryl, C₇-C₂₂-alkaryl, C₇-C₂₂-alkaryloxy, C₇-C₂₂-alkarylthio, amino, mono(C₁-C₁₂-alkyl)amino, di(C₁-C₁₂-alkyl)amino, NH(C₆-C₁₀-aryl), N(C₆-C₁₀-aryl)₂, hetaryl and oligohetaryl, where hetaryl and the hetaryl groups of oligohetaryl may each independently be unsubstituted or substituted by 1, 2, 3 or 4 radicals selected from C₁-C₁₂-alkyl and C₁-C₁₂-alkoxy.

In the compounds of the general formula (I), the R¹ to R¹² radicals are preferably each independently selected from hydrogen, C₁-C₁₂-alkyl, C₁-C₁₂-alkoxy, phenyl, naphthyl, phenyloxy, naphthyloxy and oligothiophenyl, where phenyl, naphthyl, phenyloxy, naphthyloxy and oligothiophenyl are unsubstituted or have 1 or 2 substituents which are selected from C₁-C₁₂-alkyl and C₁-C₁₂-alkoxy.

In the compounds of the general formula (I), 0, 1, 2, 3 or 4 of the R¹ to R¹² radicals preferably have a definition other than hydrogen. In a specific embodiment, in the compounds of the general formula (I), 2 of the R¹ to R¹² radicals have a definition other than hydrogen.

In the compounds of the general formula (I), at least one of the R¹, R⁵, R⁸ and R⁹ radicals has a definition other than hydrogen.

Preference is given to compounds of the general formula (I.1)

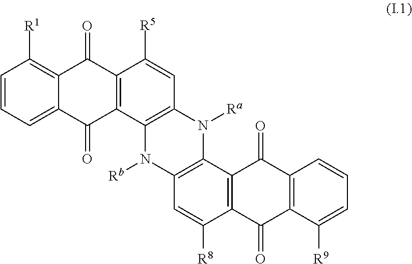

where
R^{a} and R^{b} are each independently selected from hydrogen, deuterium, C₁-C₆-alkyl, phenyl and naphthyl,
R¹ and R⁹ are each independently selected from phenyl, phenyloxy, phenylthio, naphthyl, naphthyloxy, naphthylthio, (C₁-C₁₂-alkyl)phenyl, (C₁-C₁₂-alkyl)phenyloxy, (C₁-C₁₂-alkyl)phenylthio, (C₁-C₁₂-alkyl)naphthyl, (C₁-C₁₂-alkyl)naphthyloxy and (C₁-C₁₂-alkyl)naphthylthio,
R⁵ and R⁸ are each independently selected from hydrogen, hydroxyl and C₁-C₁₂-alkoxy.

Examples of indanthrene compounds (I) which are preferentially suitable for use in organic solar cells are shown below:

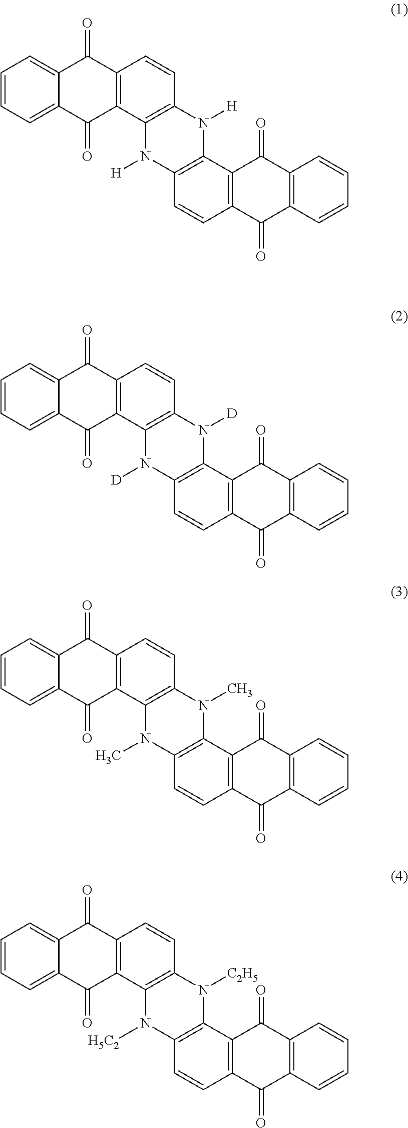

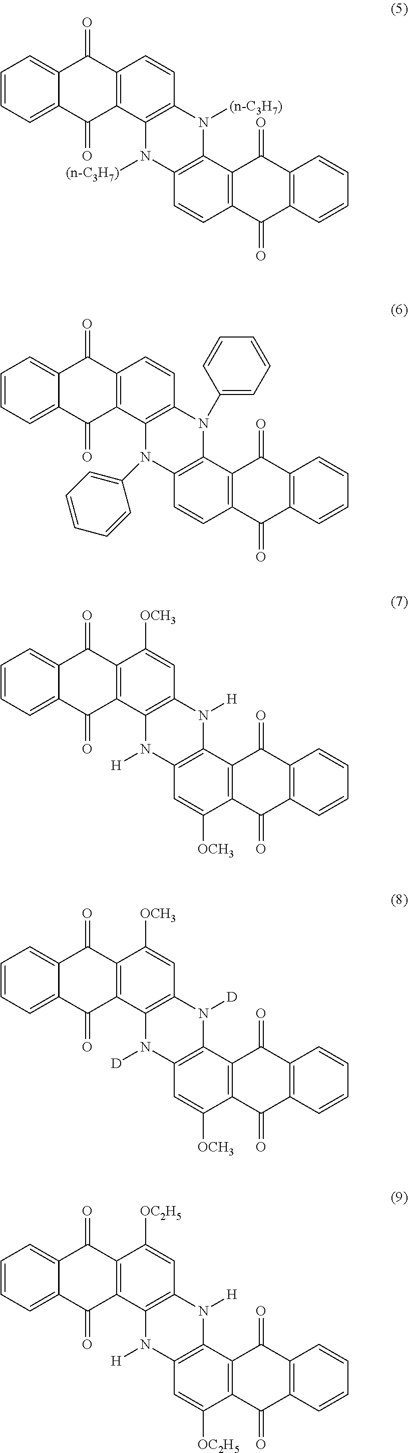

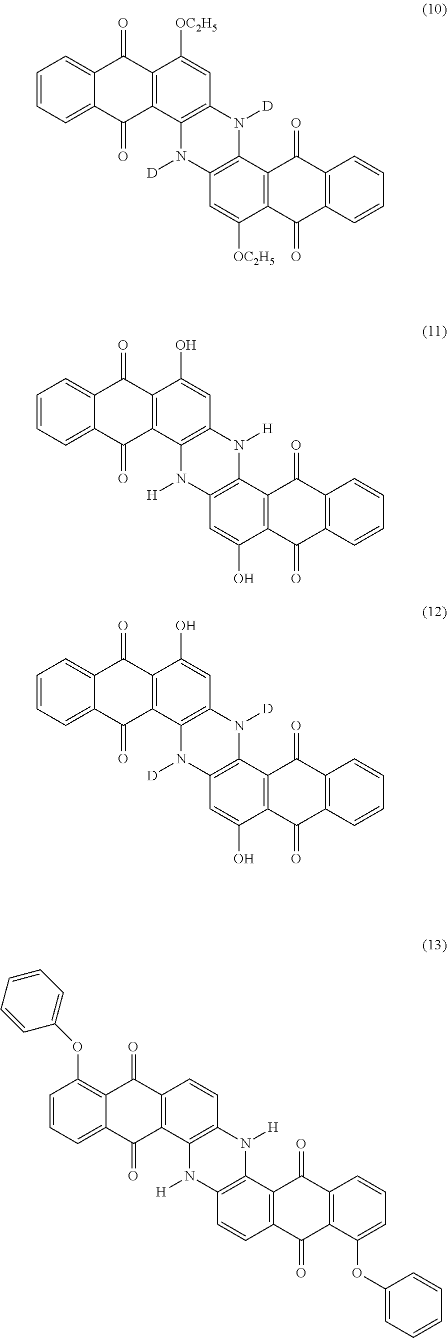

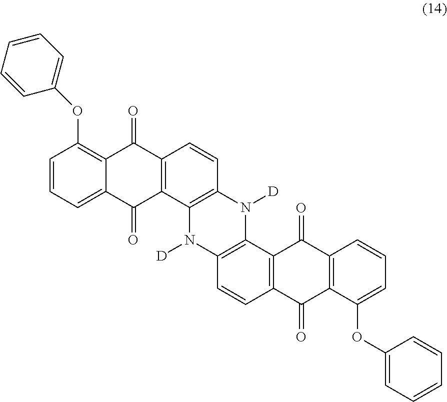

The indanthrene compounds (I) used in the inventive solar cells can be prepared by customary processes known to those skilled in the art.

The unsubstituted indanthrene (indanthrene blue, indanthrone blue) is commercially available, for example, under the Paliogen® Blue L 6480 name from BASF SE. Many other indanthrene derivatives are also commercially available.

It is possible to prepare compounds partly or fully deuterated on the nitrogen atoms from the corresponding protonated compounds by reacting with D₂SO₄ and then precipitating with D₂O. According to the desired degree of deuteration, this can be repeated once or more than once.

It is possible to prepare compounds substituted on the nitrogen atoms from the corresponding protonated compounds by customary processes.

For alkylation, it is possible to use the alkylating agents customary for this purpose, such as alkylhalides, alkylsulfates or alkylsulfonates (e.g. tosylates).

A suitable process for arylating the corresponding protonated compounds works by catalytic coupling with aryl halides in the manner of a C—N Ullmann coupling. For instance, it is possible to arylate indanthrene compounds with aryl bromides such as bromobenzene. Suitable catalysts are copper catalysts, such as CuI and Cu(I) acetate.

Before use in an organic solar cell, the indanthrene compound can be subjected to purification. The purification can be effected by customary methods known to those skilled in the art, such as separation on suitable stationary phases, sublimation, extraction, distillation, recrystallization or a combination of at least two of these measures. Each purification may have a one-stage or multistage configuration. Individual purifying operations can be repeated twice or more. Different purifying operations can be combined with one another.

In a specific embodiment, the purification comprises a column chromatography method. To this end, the starting material present in a solvent or solvent mixture can be subjected to a separation or filtration on silica gel. Finally, the solvent is removed, for example by evaporation under reduced pressure. Suitable solvents are aromatics such as benzene, toluene, xylene, mesitylene, chlorobenzene or dichlorobenzene, hydrocarbons and hydrocarbon mixtures, such as pentane, hexane, ligroin and petroleum ether, halogenated hydrocarbons such as chloroform or dichloromethane, and mixtures of the solvents mentioned. For chromatography, it is also possible to use a gradient of at least two different solvents, for example a toluene/petroleum ether gradient.

In a further specific embodiment, the purification comprises a sublimation. This may preferably be a fractional sublimation. For fractional sublimation, it is possible to use a temperature gradient in the sublimation and/or the deposition of the substituted indanthrene. In addition, the purification can be effected by sublimation with the aid of a carrier gas stream. Suitable carrier gases are inert gases, for example nitrogen, argon or helium. The gas stream laden with the compound can subsequently be passed into a separating chamber. Suitable separating chambers may have a plurality of separation zones which can be operated at different temperatures. Preference is given, for example, to a so-called three-zone sublimation apparatus. A further process and an apparatus for fractional sublimation are described in U.S. Pat. No. 4,036,594.

Organic solar cells generally have a layer structure and generally comprise at least the following layers: anode, photoactive layer and cathode. These layers are generally applied to a substrate suitable for this purpose. The structure of organic solar cells is described, for example, in US 2005/0098726 and US 2005/0224905.

The invention provides an organic solar cell which comprises a substrate with at least one cathode and at least one anode, and at least one compound of the general formula (I) as defined above as a photoactive material. The inventive organic solar cell comprises at least one photoactive region. A photoactive region may comprise two layers, each of which has a homogeneous composition and forms a flat donor-acceptor heterojunction. A photoactive region may also comprise a mixed layer and form a donor-acceptor heterojunction in the form of a donor-acceptor bulk heterojunction. Organic solar cells with photoactive donor-acceptor transitions in the form of a bulk heterojunction are a preferred embodiment of the invention.

Suitable substrates for organic solar cells are, for example, oxidic materials, polymers and combinations thereof. Preferred oxidic materials are selected from glass, ceramic, SiO₂, quartz, etc. Preferred polymers are selected from polyethylene terephthalates, polyolefins (such as polyethylene and polypropylene), polyesters, fluoropolymers, polyamides, polyurethanes, polyalkyl (meth)acrylates, polystyrenes, polyvinyl chlorides and mixtures and composites.

Suitable electrodes (cathode, anode) are in principle semiconductors, metal alloys, semiconductor alloys and combinations thereof. Preferred metals are those of groups 2, 8, 9, 10, 11 or 13 of the periodic table, e.g. Pt, Au, Ag, Cu, Al, In, Mg or Ca. Preferred semiconductors are, for example, doped Si, doped Ge, indium tin oxide (ITO), fluorinated tin oxide (FTO), gallium indium tin oxide (GITO), zinc indium tin oxide (ZITO), etc. Preferred metal alloys are for example alloys based on Pt, Au, Ag, Cu, etc. A specific embodiment is Mg/Ag alloys.

The material used for the electrode facing the light (the anode in a normal structure, the cathode in an inverse structure) is preferably a material at least partly transparent to the incident light. This preferably includes electrodes which have glass and/or a transparent polymer as a carrier material. Transparent polymers suitable as carriers are those mentioned above, such as polyethylene terephthalate. The electrical contact connection is generally effected by means of metal layers and/or transparent conductive oxides (TCOs). These preferably include ITO, doped ITO, FTO (fluorine doped tin oxide), AZO (aluminum doped tin oxide), ZnO, TiO₂, Ag, Au, Pt. Particular preference is given to ITO for contact connection. For electrical contact connection, it is also possible to use a conductive polymer, for example a poly-3,4-alkylenedioxy-thiophene, e.g. poly-3,4-ethyleneoxythiophene (PEDOT).

The electrode facing the light is configured such that it is sufficiently thin to bring about only minimal light absorption but thick enough to enable good charge transport of the extracted charge carriers. The thickness of the electrode layer (without carrier material) is preferably within a range from 20 to 200 nm.

In a specific embodiment, the material used for the electrode facing away from the light (the cathode in a normal structure, the anode in an inverse structure) is a material which at least partly reflects the incident light. This includes metal films, preferably of Ag, Au, Al, Ca, Mg, In, and mixtures thereof. Preferred mixtures are Mg/Al. The thickness of the electrode layer is preferably within a range from 50 to 300 nm.

The photoactive region comprises or consists of at least one layer which comprises at least one indanthrene compound of the general formula (I) as defined above. In addition, the photoactive region may have one or more further layer(s). These are, for example, selected from
layers with electron-conducting properties (electron transport layer, ETL),
layers which comprise a hole-conducting material (hole transport layer, HTL), which need not absorb any radiation,
exciton- and hole-blocking layers (e.g. EBLs), which must not absorb, and
multiplication layers.

Suitable materials for these layers are described in detail hereinafter.

Suitable exciton- and hole-blocking layers are described, for example, in U.S. Pat. No. 6,451,415. Suitable materials for exciton-blocking layers are, for example, bathocuproin (BCP), 4,4′,4″-tris[3-methylphenyl-N-phenylamino]triphenylamine (m-MTDATA) or polyethylenedioxythiophene (PEDOT).

The inventive solar cells comprise at least one photoactive donor-acceptor heterojunction. Optical excitation of an organic material generates excitons. In order that a photocurrent occurs, the electron-hole pair has to be separated, typically at a donor-acceptor interface between two unlike contact materials. At such an interface, the donor material forms a heterojunction with an acceptor material. When the charges are not separated, they can recombine in a process also known as “quenching”, either radiatively by the emission of light of a lower energy than the incident light or nonradiatively by generation of heat. Both processes are undesired. According to the invention, at least one substituted indanthrene of the general formula (I) can be used as a charge generator (donor). In combination with an appropriate electron acceptor material (ETM, electron transport material), radiative excitation is followed by a rapid electron transfer to the ETM. Inventive ETMs are C60 and other fullerenes.

In a first embodiment, the heterojunction has a flat configuration (see: Two layer organic photovoltaic cell, C. W. Tang, Appl. Phys. Lett., 48 (2), 183-185 (1986) or N. Karl, A. Bauer, J. Holzäpfel, J. Marktanner, M. Möbus, F. Stölzle, Mol. Cryst. Liq. Cryst., 252, 243-258 (1994).).

In a second preferred embodiment, the heterojunction is configured as a bulk (mixed) heterojunction, also referred to as an interpenetrating donor-acceptor network. Organic photovoltaic cells with a bulk heterojunction are described, for example, by C. J. Brabec, N. S. Sariciftci, J. C. Hummelen in Adv. Funct. Mater., 11 (1), 15 (2001) or by J. Xue, B. P. Rand, S. Uchida and S. R. Forrest in J. Appl. Phys. 98, 124903 (2005). Bulk heterojunctions are discussed in detail hereinafter.

The compounds of the formula (I) can be used as a photoactive material in cells with MiM, pin, pn, Mip or Min structure (M=metal, p=p-doped organic or inorganic semiconductor, n=n-doped organic or inorganic semiconductor, i=intrinsically conductive system of organic layers; see, for example, J. Drechsel et al., Org. Electron., 5 (4), 175 (2004) or Maennig et al., Appl. Phys. A 79, 1-14 (2004)).

The compounds of the formula (I) can also be used as a photoactive material in tandem cells. Suitable tandem cells are described, for example, by P. Peumans, A. Yakimov, S. R. Forrest in J. Appl. Phys, 93 (7), 3693-3723 (2003) (see also U.S. Pat. No. 4,461,922, U.S. Pat. No. 6,198,091 and U.S. Pat. No. 6,198,092) and are described in detail hereinafter. The use of indanthrene compounds of the general formula (I) in tandem cells is a preferred embodiment of the invention.

The compounds of the formula (I) can also be used as a photoactive material in tandem cells which are constructed from two or more than two stacked MiM, pin, Mip or Min structures (see DE 103 13 232.5 and J. Drechsel et al., Thin Solid Films, 451452, 515-517 (2004)).

The layer thickness M, n, i and p layers is typically within a range from 10 to 1000 nm, more preferably from 10 to 400 nm. The layers which form the solar cell can be produced by customary processes known to those skilled in the art. These include vapor deposition under reduced pressure or in an inert gas atmosphere, laser ablation or solution or dispersion processing methods such as spincoating, knifecoating, casting methods, spray application, dipcoating or printing (e.g. inkjet, flexographic, offset, gravure; intaglio, nanoimprinting). In a specific embodiment, the entire solar cell is produced by a gas phase deposition process.

In order to improve the efficiency of organic solar cells, it is possible to shorten the mean distance through which the exciton has to diffuse in order to arrive at the next donor-acceptor interface. To this end, it is possible to use mixed layers of donor material and acceptor material which form an interpenetrating network in which internal donor-acceptor heterojunctions are possible. This bulk heterojunction is a specific form of the mixed layer, in which the excitons generated need only travel a very short distance before they arrive at a domain boundary, where they are separated.

In a preferred embodiment, the photoactive donor-acceptor transitions in the form of a bulk heterojunction are produced by a gas phase deposition process (physical vapor deposition, PVD). Suitable processes are described, for example, in US 2005/0227406, to which reference is made here. To this end, an indanthrene compound of the general formula (I) and a complementary semiconductor material can be subjected to a gas phase deposition in the manner of a cosublimation. PVD processes are performed under high-vacuum conditions and comprise the following steps: evaporation, transport, deposition. The deposition is effected preferably at a pressure within a range from about 10⁻² mbar to 10⁻⁷ mbar, for example from 10⁻⁵ to 10⁻⁷ mbar. The deposition rate is preferably within a range from 0.01 to 10 nm/s. The deposition can be effected in an inert gas atmosphere, for example under nitrogen, helium or argon. The temperature of the substrate during the deposition is preferably within a range from −100 to 300° C., more preferably from −50 to 250° C.

The other layers of the organic solar cell can be produced by known processes. These include vapor deposition under reduced pressure or in an inert gas atmosphere, laser ablation, or solution or dispersion processing methods such as spincoating, knifecoating, casting methods, spray application, dipcoating or printing (e.g. inkjet, flexographic, offset, gravure; intaglio, nanoimprinting). In a specific embodiment, the entire solar cell is produced by a gas phase deposition process.

The photoactive layer (homogeneous layer or mixed layer) can be subjected to a thermal treatment directly after production thereof or after production of further layers which form the solar cell. Such a heat treatment can in many cases further improve the morphology of the photoactive layer. The temperature is preferably within a range from about 60° C. to 300° C. The treatment time is preferably within a range from 1 minute to 3 hours. In addition or alternatively to a thermal treatment, the photoactive layer (mixed layer) can be subjected to a treatment with a solvent-containing gas directly after production thereof or after production of further layers which form the solar cell. In a suitable embodiment, saturated solvent vapors in air are used at ambient temperature. Suitable solvents are toluene, xylene, chloroform, N-methylpyrrolidone, dimethylformamide, ethyl acetate, chlorobenzene, dichloromethane and mixtures thereof. The treatment time is preferably within a range from 1 minute to 3 hours.

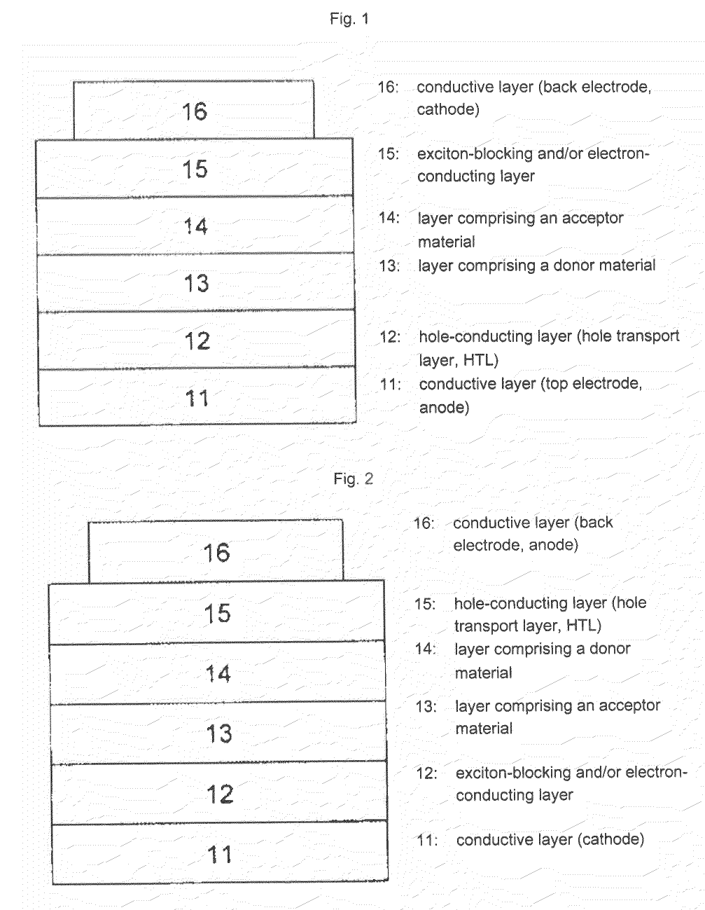

In a preferred embodiment, the inventive solar cells are present as an individual cell with flat heterojunction normal structure. FIG. 1 shows an inventive solar cell with normal structure. In a specific embodiment, the cell has the following structure:
an at least partly transparent conductive layer (top electrode, anode) (11)
a hole-conducting layer (hole transport layer, HTL) (12)
a layer which comprises a donor material (13)
a layer which comprises an acceptor material (14)
an exciton-blocking and/or electron-conducting layer (15)
a second conductive layer (back electrode, cathode) (16)

The donor material preferably comprises at least one compound of the formula (I) or consists of a compound of the formula (I). According to the invention, the acceptor material comprises at least one fullerene or fullerene derivative, or consists of a fullerene or fullerene derivative. The acceptor material preferably comprises C60 or PCBM ([6,6]-phenyl-C61-butyric acid methyl ester).

The essentially transparent conductive layer (11) (anode) comprises a carrier, such as glass or a polymer (e.g. polyethylene terephthalate) and a conductive material, as described above. Examples include ITO, doped ITO, FTO, ZnO, AZO, etc. The anode material can be subjected to a surface treatment, for example with UV light, ozone, oxygen plasma, Br₂, etc. The layer (11) should be sufficiently thin to enable maximum light absorption, but also sufficiently thick to ensure good charge transport. The layer thickness of the transparent conductive layer (11) is preferably within a range from 20 to 200 nm.

The solar cell with normal structure according to FIG. 1 optionally has a hole-conducting layer (HTL). This layer comprises at least one hole-conducting material (hole transport material, HTM). Layer (12) may be an individual layer of essentially homogeneous composition or may comprise two or more than two sublayers.

Hole-conducting materials (HTM) suitable for forming layers with hole-conducting properties (HTL) preferably comprise at least one material with high ionization energy. The ionization energy is preferably at least 5.0 eV, more preferably at least 5.5 eV. The materials may be organic or inorganic materials. Organic materials suitable for use in a layer with hole-conducting properties are preferably selected from poly(3,4-ethylenedioxythiophene) poly(styrenesulfonate) (PEDOT-PSS), Ir-DPBIC (tris-N,N′-diphenylbenzimidazol-2-ylideneiridium(III)), N,N′-diphenyl-N,N′-bis(3-methylphenyl)-1,1′-diphenyl-4,4′-diamine (α-NPD), 2,2′,7,7′-tetrakis(N,N-di-p-methoxyphenylamine)-9,9′-spirobifluorene (spiro-MeOTAD), etc. and mixtures thereof. The organic materials may, if desired, be doped with a p-dopant which has a LUMO within the same range as or lower than the HOMO of the hole-conducting material. Suitable dopants are, for example, 2,3,5,6-tetrafluoro-7,7,8,8-tetracyanoquinodimethane (F₄TCNQ), WO₃, MoO₃, etc. Inorganic materials suitable for use in a layer with hole-conducting properties are preferably selected from WO₃, MoO₃, etc.

If present, the thickness of the layers with hole-conducting properties is preferably within a range from 5 to 200 nm, more preferably 10 to 100 nm.

Layer (13) comprises at least one compound of the general formula (I). The thickness of the layer should be sufficient to absorb a maximum amount of light, but thin enough to enable effective dissipation of the charge. The thickness of the layer (13) is preferably within a range from 5 nm to 1 μm, more preferably from 5 to 80 nm.

Layer (14) comprises at least one acceptor material. According to the invention the acceptor material comprises at least one fullerene or fullerene derivative. The thickness of the layer should be sufficient to absorb a maximum amount of light, but thin enough to enable effective dissipation of the charge. The thickness of the layer (14) is preferably within a range from 5 nm to 1 μm, more preferably from 5 to 80 nm.

The solar cell with normal structure according to FIG. 1 optionally comprises an exciton-blocking and/or electron-conducting layer (15) (EBL/ETL). Suitable materials for exciton-blocking layers generally have a greater band gap than the materials of layer (13) and/or (14). They are firstly capable of reflecting excitons and secondly enable good electron transport through the layer. The materials for the layer (15) may comprise organic or inorganic materials. Suitable organic materials are preferably selected from 2,9-dimethyl-4,7-diphenyl-1,10-phenanthroline (BCP), 4,7-diphenyl-1,10-phenanthroline (Bphen), 1,3-bis[2-(2,2bipyridin-6-yl)-1,3,4-oxadiazo-5-yl]benzene (BPY-OXD), etc. The organic materials may, if desired, be doped with an n-dopant which has a HOMO within the same range as or lower than the LUMO of the electron-conducting material. Suitable dopants are, for example, Cs₂CO₃, Pyronin B (PyB), Rhodamine B, cobaltocenes, etc. Inorganic materials suitable for use in a layer with electron-conducting properties are preferably selected from ZnO, etc. If present, the thickness of the layer (15) is preferably within a range from 5 to 500 nm, more preferably 10 to 100 nm.

Layer 16 is the cathode and preferably comprises at least one compound with low work function, more preferably a metal such as Ag, Al, Mg, Ca, etc. The thickness of the layer (16) is preferably within a range from about 10 nm to 10 μm, e.g. 10 nm to 60 nm.

In a preferred embodiment, the inventive solar cells are present as an individual cell with a flat heterojunction and inverse structure. FIG. 2 shows a solar cell with inverse structure. In a specific embodiment, the cell has the following structure:
an at least partly transparent conductive layer (cathode) (11)
an exciton-blocking and/or electron-conducting layer (12)
a layer which comprises an acceptor material (13)
a layer which comprises a donor material (14)
a hole-conducting layer (hole transport layer, HTL) (15)
a second conductive layer (back electrode, anode) (16)

With regard to suitable and preferred materials for the layers (11) to (16), reference is made to the above remarks regarding the corresponding layers in solar cells with normal structure.

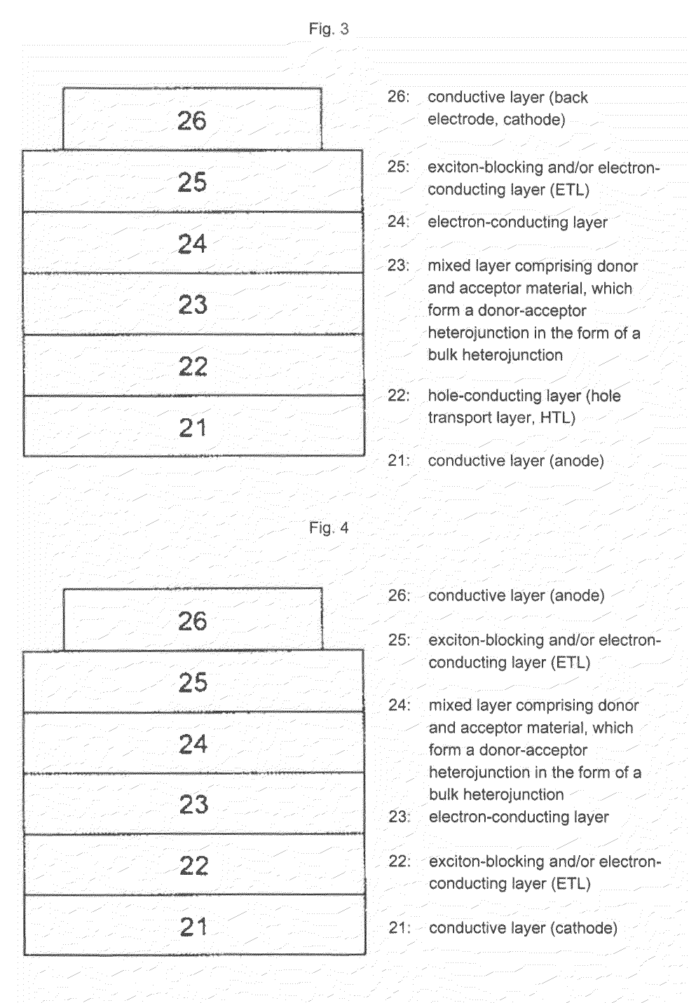

In a further preferred embodiment, the inventive solar cells are present as an individual cell with normal structure and have a bulk heterojunction. FIG. 3 shows a solar cell with a bulk heterojunction. In a specific embodiment, the cell has the following structure:
an at least partly transparent conductive layer (anode) (21)
a hole-conducting layer (hole transport layer, HTL) (22)
a mixed layer which comprises a donor material and an acceptor material, which form a donor-acceptor heterojunction in the form of a bulk heterojunction (23)
an electron-conducting layer (24)
an exciton-blocking and/or electron-conducting layer (25)
a second conductive layer (back electrode, cathode) (26)

The layer (23) comprises at least one indanthrene compound of the general formula (I) as a photoactive material, especially as a donor material. The layer (23) additionally comprises preferably at least one fullerene or fullerene derivative as an acceptor material. The layer (23) comprises especially C60 or PCBM ([6,6]-phenyl-C61-butyric acid methyl ester) as an acceptor material.

With regard to layer (21), reference is made completely to the above remarks regarding layer (11).

With regard to layer (22), reference is made completely to the above remarks regarding layer (12).

Layer (23) is a mixed layer which comprises at least one compound of the general formula (I) as a donor material, i.e. fullerene or a fullerene derivative. In addition, layer (23) comprises at least one acceptor material. As described above, the layer (23) can be produced by coevaporation or by solution processing using customary solvents. The mixed layer comprises preferably 10 to 90% by weight, more preferably 20 to 80% by weight, of at least one compound of the general formula (I), based on the total weight of the mixed layer. The mixed layer comprises preferably 10 to 90% by weight, more preferably 20 to 80% by weight, of at least one acceptor material, based on the total weight of the mixed layer. The thickness of the layer (23) should be sufficient to absorb a maximum amount of light, but thin enough to enable effective dissipation of the charge. The thickness of the layer (23) is preferably within a range from 5 nm to 1 μm, more preferably from 5 to 200 nm, especially 5 to 80 nm.

The solar cell with a bulk heterojunction according to FIG. 3 comprises an electron-conducting layer (24) (ETL). This layer comprises at least one electron transport material (ETM). Layer (24) may be a single layer of essentially homogeneous composition or may comprise two or more than two sublayers. Suitable materials for electron-conducting layers generally have a low work function or ionization energy. The ionization energy is preferably not more than 3.5 eV. Suitable organic materials are preferably selected from the aforementioned fullerenes and fullerene derivatives, 2,9-dimethyl-4,7-diphenyl-1,10-phenanthroline (BCP), 4,7-diphenyl-1,10-phenanthroline (Bphen), 1,3-bis[2-(2,2bipyridin-6-yl)-1,3,4-oxadiazo-5-yl]benzene (BPY-OXD), etc. The organic materials used in layer (24) may, if desired, be doped with an n-dopant which has a HOMO within the same range as or lower than the LUMO of the electron-conducting material. Suitable dopants are, for example, Cs₂CO₃, Pyronin B (PyB), Rhodamine B, cobaltocenes, etc. The thickness of the layer (23) is, if present, preferably within a range from 1 nm to 1 μm, particularly 5 to 60 nm.

With regard to layer (25), reference is made completely to the above remarks regarding layer (15).

With regard to layer (26), reference is made completely to the above remarks regarding layer (16).

The solar cell with a donor-acceptor heterojunction in the form of a bulk heterojunction can be produced by a gas phase deposition process as described above. With regard to deposition rates, substrate temperature during the deposition and thermal aftertreatment, reference is made to the above remarks.

In a further preferred embodiment, the inventive solar cells are present as an individual cell with inverse structure and have a bulk heterojunction. FIG. 4 shows a solar cell with a bulk heterojunction and inverse structure.

In a further particularly preferred embodiment, the inventive solar cell is a tandem cell.

A tandem cell consists of two or more than two (e.g. 3, 4, 5, etc.) subcells. A single subcell, some of the subcells or all subcells may have photoactive donor-acceptor heterojunctions. Each donor-acceptor-heterojunction may be in the form of a flat heterojunction or in the form of a bulk heterojunction. Preferably, at least one of the donor-acceptor heterojunctions is in the form of a bulk heterojunction. According to the invention, the photoactive layer of at least one subcell comprises an indanthrene compound of the general formula (I). Preferably, the photoactive layer of at least one subcell comprises an indanthrene compound of the general formula (I) and at least one fullerene or fullerene derivative. More preferably, the semiconductor mixture used in the photoactive layer of at least one subcell consists of an indanthrene compound of the general formula (I) and C₆O or [6,6]-phenyl-C61-butyric acid methyl ester.

The subcells which form the tandem cell may be connected in parallel or in series. The subcells which form the tandem cell are preferably connected in series. There is preferably an additional recombination layer in each case between the individual subcells. The individual subcells have the same polarity, i.e. generally either only cells with normal structure or only cells with inverse structure are combined with one another.

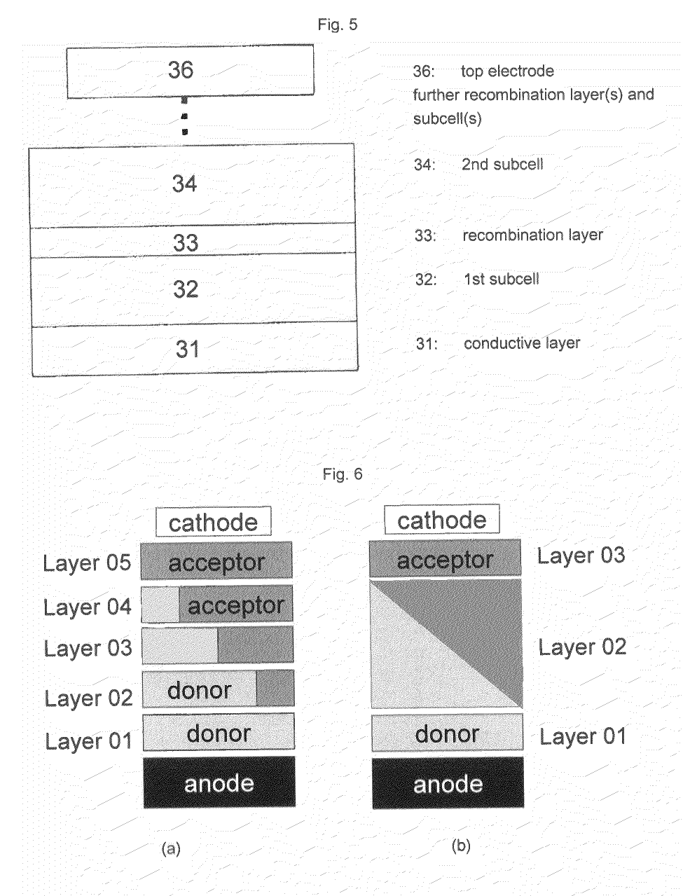

FIG. 5 shows the basic structure of an inventive tandem cell. Layer 31 is a transparent conductive layer. Suitable materials are those specified above for the individual cells.

Layers 32 and 34 constitute subcells. “Subcell” refers here to a cell as defined above without cathode and anode. The subcells may, for example, either all have an indanthrene compound of the general formula (I) used in accordance with the invention in the photoactive layer (preferably in combination with a fullerene or fullerene derivative, especially C60) or have other combinations of semiconductor materials, for example C60 with zinc phthalocyanine, C60 with oligothiophene (such as DCV5T). In addition, individual subcells may also be configured as dye-sensitized solar cells or polymer cells. In all cases, preference is given to a combination of materials which exploit different regions of the spectrum of the incident light, for example of natural sunlight. For instance, the combination of indanthrene compound of the general formula (I) and fullerene or fullerene derivative used in accordance with the invention absorbs in the long-wave region of sunlight. Dibenzoperiflanthene(DBP)-C60 absorbs primarily in the range from 400 nm to 600 nm. Zinc phthalocyanine-C60 cells absorb primarily in the range from 600 nm to 800 nm. Thus, a tandem cell composed of a combination of these subcells should absorb radiation in the range from 400 nm to 800 nm. Suitable combination of subcells should thus allow the spectral range utilized to be extended. For optimal performance properties, optical interference should be considered. For instance, subcells which absorb at relatively short wavelengths should be arranged closer to the metal top contact than subcells with longer-wave absorption.

With regard to layer (31), reference is made completely to the above remarks regarding layers (11) and (21).

With regard to layers (32) and (34), reference is made completely to the above remarks regarding layers (12) to (15) for flat heterojunctions and (22) to (25) for bulk heterojunctions.

Layer 33 is a recombination layer. Recombination layers enable the charge carriers from one subcell to recombine with those of an adjacent subcell. Small metal clusters are suitable, such as Ag, Au or combinations of highly n- and p-doped layers. In the case of metal clusters, the layer thickness is preferably within a range from 0.5 to 5 nm. In the case of highly n- and p-doped layers, the layer thickness is preferably within a range from 5 to 40 nm. The recombination layer generally connects the electron-conducting layer of a subcell to the hole-conducting layer of an adjacent subcell. In this way, further cells can be combined to form the tandem cell.

Layer 36 is the top electrode. The material depends on the polarity of the subcells. For subcells with normal structure, preference is given to using metals with a low work function, such as Ag, Al, Mg, Ca, etc. For subcells with inverse structure, preference is given to using metals with a high work function, such as Au or Pt, or PEDOT-PSS.

In the case of subcells connected in series, the overall voltage corresponds to the sum of the individual voltages of all subcells. The overall current, in contrast, is limited by the lowest current of one subcell. For this reason, the thickness of each subcell should be optimized such that all subcells have essentially the same current.

Examples of different kinds of donor-acceptor heterojunctions are a donor-acceptor double layer with a flat heterojunction, or the heterojunction is configured as a hybrid planar-mixed heterojunction or gradient bulk heterojunction or annealed bulk heterojunction.

The production of a hybrid planar-mixed heterojunction is described in Adv. Mater. 17, 66-70 (2005). In this structure, mixed heterojunction layers which were formed by simultaneous evaporation of acceptor and donor material are present between homogeneous donor and acceptor material.

In a specific embodiment of the present invention, the donor-acceptor-heterojunction is in the form of a gradient bulk heterojunction. In the mixed layers composed of donor and acceptor materials, the donor-acceptor ratio changes gradually. The form of the gradient may be stepwise (FIG. 6(a)) or linear (FIG. 6(b)). In FIG. 6(a), the layer 01 consists of 100% donor material, layer 02 has a donor/acceptor ratio>1, layer 03 has a donor/acceptor ratio=1, layer 04 has a donor/acceptor ratio<1, and layer 05 consists of 100% acceptor material. In FIG. 6(b), layer 01 consists of 100% donor material, layer 02 has a decreasing ratio of donor/acceptor, i.e. the proportion of donor material decreases in a linear manner in the direction of layer 03, and layer 03 consists of 100% acceptor material. The different donor-acceptor ratios can be controlled by means of the deposition rate of each and every material. Such structures can promote the percolation path for charges.

In a further specific embodiment of the present invention, the donor-acceptor-heterojunction is configured as an annealed bulk heterojunction; see, for example, Nature 425, 158-162, 2003. The process for producing such a solar cell comprises an annealing step before or after the metal deposition. As a result of the annealing, donor and acceptor materials can separate, which leads to more extended percolation paths.

In a further specific embodiment of the present invention, the organic solar cells are produced by organic vapor phase deposition, either with a flat or a controlled heterojunction architecture. Solar cells of this type are described in Materials, 4, 2005, 37.

In a specific embodiment, at least one substituted indanthrene of the general formula (I) is used as the sole electron donor material.

The inventive organic solar cells comprise at least one photoactive region which comprises at least one indanthrene compound as a donor, which is in contact with at least one fullerene compound as an acceptor. Fullerenes and fullerene derivatives, preferably selected from C₆₀, C₇₀, C₈₄, phenyl-C₆₁-butyric acid methyl ester ([60]PCBM), phenyl-C₇₁-butyric acid methyl ester ([71]PCBM), phenyl-C₈₄-butyric acid methyl ester ([84]PCBM), phenyl-C₆₁-butyric acid butyl ester ([60]PCBB), phenyl-C₆₁-butyric acid octyl ester ([60]PCBO), thienyl-C₆₁-butyric acid methyl ester ([60]ThCBM) and mixtures thereof. Particular preference is given to C₆₀, [60]PCBM and mixtures thereof.

In addition to indanthrene compounds and fullerenes, the semiconductor materials listed hereinafter are suitable in principle for use in the inventive solar cells. They serve as donors or acceptors for subcells of a tandem cell, which are combined with an indanthrene/fullerene subcell used in accordance with the invention.

Suitable further semiconductors are phthalocyanines. These include phthalocyanines which are nonhalogenated or which bear 1 to 16 halogen atoms. The phthalocyanines may be metal-free or contain a divalent metal or a metal atom-containing group. Preference is given to phthalocyanines based on zinc, copper, iron, titanyloxy, vanadyloxy, etc. Particular preference is given to copper phthalocyanines, zinc phthalocyanines, metal-free phthalocyanines. In a specific embodiment, a halogenated phthalocyanine is used. These include:

2,6,10,14-tetrafluorophthalocyanines, e.g. copper 2,6,10,14-tetrafluorophthalocyanine and zinc 2,6,10,14-tetrafluorophthalocyanine;

1,5,9,13-tetrafluorophthalocyanines, e.g. copper 1,5,9,13-tetrafluorophthalocyanines and zinc 1,5,9,13-tetrafluorophthalocyanines;

2,3,6,7,10,11,14,15-octafluorophthalocyanine, e.g. copper 2,3,6,7,10,11,14,15-octafluorophthalocyanine and zinc 2,3,6,7,10,11,14,15-octafluorophthalocyanine; phthalocyanines which are suitable as acceptors are, for example, hexadecachlorophthalocyanines and hexadecafluorophthalocyanines, such as copper hexadecachlorophthalocyanine, zinc hexadecachlorophthalocyanine, metal-free hexadecachlorophthalocyanine, copper hexadecafluorophthalocyanine, hexadecafluorophthalocyanine or metal-free hexadecafluorophthalocyanine.

Suitable further semiconductors, which are predominantly suitable as acceptors, are rylenes. In the context of the invention, rylenes are generally understood to mean compounds with a molecular structure of peri-linked naphthalene units. According to the number of naphthalene units, the compounds may, for example, be perylenes (n=2), terrylenes (n=3), quaterrylenes (n=4) or higher rylenes. Accordingly, they may be perylenes, terrylenes or quaterrylenes of the following formulae.

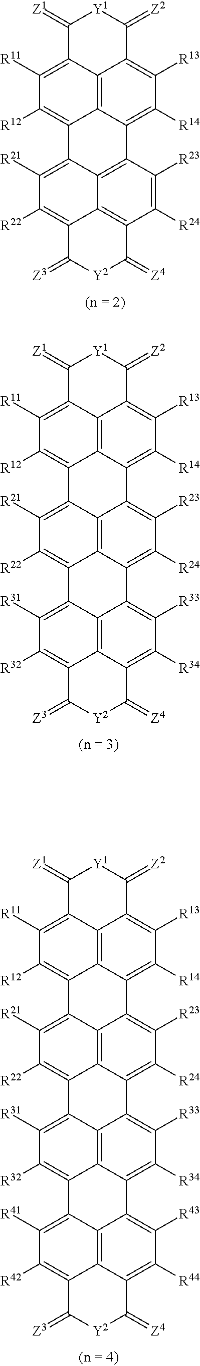

in which

the Rⁿ¹, Rⁿ², Rⁿ³ and Rⁿ⁴ radicals for n=1 to 4 are each independently hydrogen, halogen or groups other than halogen,

Y¹ is O or NR^{a} where R^{a} is hydrogen or an organyl radical,

Y² is O or NR^{b} where R^{b} is hydrogen or an organyl radical,

Z¹, Z², Z³ and Z⁴ are each O,

where, in the case that Y¹ is NR^{a}, one of the Z¹ and Z² radicals may also be NR^{c}, where the R^{a} and R^{c} radicals together are a bridging group having 2 to 5 atoms between the flanking bonds, and

where, in the case that Y² is NR^{b}, one of the Z³ and Z⁴ radicals may also be NR^{d}, where the R^{b} and R^{d} radicals together are a bridging group having 2 to 5 atoms between the flanking bonds.

Suitable rylenes are, for example, described in WO 2007/074137, WO 2007/093643 and WO 2007/116001, to which reference is made here.

Also suitable are the following donor-semiconductor materials, which can be used, for example, in a tandem cell, as described hereinafter, in a further subcell instead of the indanthrene compounds (I).

Semiconductors suitable as donors are porphyrins, for example 5,10,15,20-tetra(3-pyridyl)porphyrin (TpyP), or else tetrabenzoporphyrins, for example metal-free tetrabenzoporphyrin, copper tetrabenzoporphyrin or zinc tetrabenzoporphyrin. Especially preferred are tetrabenzoporphyrins. These can be processed from solution as soluble precursors and converted to the photoactive component by thermolysis on the substrate.

Further semiconductors suitable as donors are acenes. These are preferably selected from in each case unsubstituted or substituted anthracene, tetracene or pentacene. Substituted acenes comprise preferably at least one substituent which is selected from electron-donating substituents, electron-withdrawing substituents and combinations thereof. Suitable electron-donating substituents are, for example, alkyl, alkoxy, ester, carboxylate or thioalkoxy. Suitable electron-withdrawing substituents are, for example, halogen, nitro or cyano. Preferred acenes are selected from 2,9-dialkylpentacenes, 2,10-dialkylpentacenes, 2,10-dialkoxypentacenes, 1,4,8,11-tetraalkoxypentacenes and rubrene (5,6,11,12-tetraphenylnaphthacene). Suitable substituted pentacenes are described in US 2003/0100779 and U.S. Pat. No. 6,864,396, to which reference is made here. A particularly preferred acene is rubrene.

Further semiconductors suitable as donors are liquid-crystalline materials (LC materials). These are preferably selected from coronenes and triphenylenes. Preferred liquid-crystalline materials are hexabenzocoronene (HBC-PhC₁₂), coronenediimides, 2,3,6,7,10,11-hexahexylthiotriphenylene (HTT₆), 2,3,6,7,10,11-hexakis-(4-n-nonylphenyl)triphenylene (PTP9) or 2,3,6,7,10,11-hexakis(undecyloxy)triphenylene (HAT₁₁). Particular preference is given to liquid-crystalline materials which are discotic.

Further semiconductors suitable as donors are thiophene compounds. These are preferably selected from thiophenes, oligothiophenes and substituted derivatives thereof. Suitable oligothiophenes are quaterthiophenes, quinquethiophenes, sexithiophenes, α,ω-di(C₁-C₈)-alkyloligothiophenes, such as α,ω-dihexylquaterthiophenes, α,ω-dihexylquinquethiophenes and α,ω-dihexylsexithiophenes, poly(alkylthiophenes) such as poly(3-hexylthiophene), bis(dithienothiophenes), anthradithiophenes and dialkylanthradithiophenes such as dihexylanthradithiophene, phenylene-thiophene (P-T) oligomers and derivatives thereof, especially α,ω-alkyl-substituted phenylene-thiophene oligomers.

Further thiophene compounds suitable as semiconductors are preferably selected from compounds like

α,α′-bis(2,2-dicyanovinyl)quinquethiophene (DCV5T),

(3-(4-octylphenyl)-2,2′-bithiophene) (PTOPT),

poly-3-(4′-(1,4,7-trioxaoctyl)-phenyl)thiophene (PEOPT),

(poly(3-(2′-methoxy-5′-octylphenyl)thiophene)) (POMeOPT),

poly(3-octylthiophene) (P₃OT),

poly[2,6-(4,4-bis(2-ethylhexyl)-4H-cyclopenta[2,1 b;3,4 b′]dithiophene)-4,7-(2,1,3-benzothiadiazole) (PCPDTBT), and also

poly(pyridopyrazinevinylene)-polythiophene blends, such as EHH-PpyPz, PTPTB copolymers, BBL, F₈BT, PFMO (see Brabec C., Adv. Mater., 2996, 18, 2884).

Further semiconductors suitable as donors are paraphenylenevinylene and oligomers or polymers comprising paraphenylenevinylene units. These are preferably selected from polyparaphenylenevinylene, MEH-PPV (poly(2-methoxy-5-(2′-ethylhexyloxy)-1,4-phenylenevinylene, MDMO-PPV (poly(2-methoxy-5-(3′,7′-dimethyloctyloxy)-1,4-phenylenevinylene)), PPV, CN-PPV (with various alkoxy derivatives), and also phenyleneethynylene/phenylenevinylene hybrid polymers (PPE-PPV).

Further semiconductors suitable as donors are polyfluorenes and alternating polyfluorene copolymers. These are preferably selected from

4,7-dithien-2′-yl-2,1,3-benzothiadiazole,

poly(9,9′-dioctylfluorene-co-benzothiadiazole) (F₈BT), and

poly(9,9′-dioctylfluorene-co-bis-N,N′-(4-butylphenyl)-bis-N,N′-phenyl-1,4-phenylenediamine (PFB).

Further semiconductors suitable as donors are polycarbazoles, i.e. oligomers and polymers comprising carbazole.

Further semiconductors suitable as donors are polyanilines, i.e. oligomers and polymers comprising aniline.

Further semiconductors suitable as donors are triarylamines, polytriarylamines, polycyclopentadienes, polypyrroles, polyfurans, polysiloles, polyphospholes, TPD, CBP, spiro-MeOTAD.

In a preferred embodiment, the inventive solar cell has the following layers:

ITO

indanthrene compound of the formula (I)

C60

BPhen (=4,7-diphenyl-1,10-phenanthroline)

Ag

The inventive solar cell is more preferably a tandem cell, wherein one subcell has a photoactive region which comprises at least one indanthrene compound of the formula (I) and C60.

All aforementioned semiconductors may be doped. The conductivity of semiconductors can be increased by chemical doping techniques using dopants. An organic semiconductor material may be doped with an n-dopant which has a HOMO energy level which is close to or higher than the LUMO energy level of the electron-conducting material. An organic semiconductor material may also be doped with a p-dopant which has a LUMO energy level which is close to or higher than the HOMO energy level of the hole-conducting material. In other words, in the case of n-doping an electron is released from the dopant, which acts as the donor, whereas in the case of p-doping the dopant acts as an acceptor which accepts an electron.

Suitable dopants for the indanthrene compounds used in accordance with the invention and for p-semiconductors in general are, for example, selected from WO₃, MoO₃, 2,3,5,6-tetrafluoro-7,7,8,8-tetracyanoquinodimethane (F₄-TCNQ), 3,6-difluoro-2,5,7,7,8,8-hexacyanoquinodimethane, dichlorodicyanoquinone (DDQ) or tetracyanoquinodimethane (TCNQ). A preferred dopant is 3,6-difluoro-2,5,7,7,8,8-hexacyanoquinodimethane.

Suitable dopants for the p-semiconductors used in accordance with the invention are, for example, selected from Cs₂CO₃, LiF, pyronin B (PyB), rhodamin derivatives, cobaltocenes, etc. Preferred dopants are pyronin B and rhodamin derivatives, especially rhodamin B.

The dopants are typically used in an amount of up to 10 mol%, preferably up to 5 mol %, based on the amount of the semiconductor to be doped.

The invention is illustrated in detail with reference to the nonlimiting examples which follow.

### EXAMPLES

### I) Preparation Examples

### Example 1

### Deuterated Indanthrene Blue

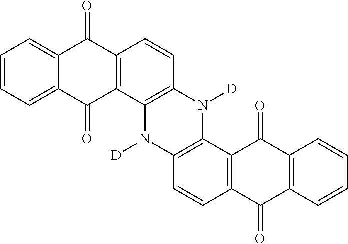

3.0 g of indanthrene blue were stirred at room temperature in 30 mol of D₂SO₄ for 20 hours. Subsequently, the solution was added to 100 ml of D₂O to precipitate the product, filtered and washed to neutrality with D₂O. This gave 2.98 g of product. This was dissolved again in 30 ml of D₂SO₄ and stirred at room temperature for 20 hours. Thereafter, 50 ml of D₂O were added dropwise, the resulting precipitate was filtered off and the residue was washed with D₂O and dried. This gave 2.9 g of deuterated product.

To produce solar cells, 2.0 g of this material were subjected three times to a gradient sublimation at 375° C./325° C./250° C. This gave 829 mg of a blue product.

### Example 2

### N,N′-Diphenylindanthrene

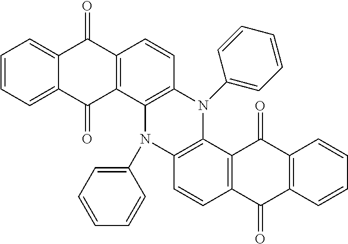

A mixture of 20.0 g (45.2 mmol) of indanthrene blue, 28.5 g (182 mmol) of bromobenzene, 19.25 g (182 mmol) of sodium carbonate, 0.2 g (3.18 mmol) of copper(I) iodide and 0.34 g (1.8 mmol) of copper(I) acetate was heated to 195° C. in 50 ml of nitrobenzene for 18 hours. The reaction mixture was cooled and filtered with suction through a filter filled with silica gel. A mixture of acetone and dichloromethane was used to elute the product. The product thus obtained was again purified by chromatography with cyclohexane/ethyl acetate (2:1). This gave 1.8 g (7%) of a blue solid.

630 mg of this product were subjected to a gradient sublimation at 265° C./200° C./150° C. This gave 160 mg of a blue material, which was used to produce solar cells.

### Example 3

### N,N′-Dimethylindanthrene

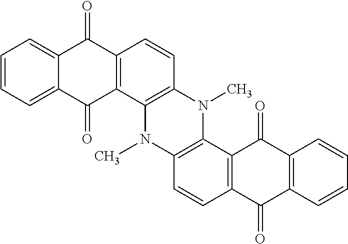

To a mixture of 160 g (856 mmol) of methyl-p-toluenesulfonate and 120 g (872 mmol) of potassium carbonate in 1.4 l of trichlorobenzene were added 40.0 g (90.4 mmol) of indanthrene blue, and the mixture was heated to reflux for 120 hours. The reaction mixture was cooled to 120° C. and filtered at this temperature. The solvent was distilled off and the crude product was purified chromatographically using silica gel with toluene/dichloromethane (10:1) and then with pure dichloromethane. This gave 4.96 g of product which was subjected to a gradient sublimation.

### Example 4

### 4,4′-Dimethoxyindanthrone

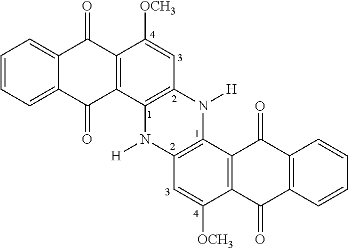

### 4.1 1-Methoxy-4-nitroanthraquinone

A mixture of 10.77 g (40 mmol) of 1-hydroxy-4-nitroanthraquinone, 3.8 g (27.5 mmol) of potassium carbonate, 9.28 g (50 mmol) of methyl p-toluenesulfonate in 60 ml of dichlorobenzene was heated to 178° C. for four hours. Subsequently, the reaction mixture was cooled and poured onto 200 ml of ice-water, and the residue of the biphasic mixture was filtered, extracted by stirring with demineralized water and dried under reduced pressure. This gave 10.5 g (93%) of a beige product, which was used without further purification in the next stage.

### 4.2 1-Methoxy-4-aminoanthraquinone

A mixture of 7.5 g (26 mmol) of 1-methoxy-4-nitroanthraquinone, 19.96 g (154 mmol) of sodium sulfide in 240 ml of water was heated under reflux for 30 minutes. The reaction mixture was cooled and added to 500 ml of saturated sodium chloride solution. The residue was filtered off with suction and washed with dilute hydrochloric acid. This gave 6.5 g (90%) of a red solid, which is used without further purification in the next stage.

### 4.3 4,4′-Dimethoxyindanthrone

A mixture of 4.0 g (16 mmol) of 1-methoxy-4-aminoanthraquinone, 12.17 g (107 mmol) of N,N′-dimethylpropyleneurea and 3.5 g of a 50% KOH solution were heated to 130° C. while introducing air. The mixture was heated until no reactant was detectable any longer in the thin-film chromatogram. The reaction mixture was cooled and poured onto water, and the residue was filtered off. The product was purified by crystallization from N-methylpyrrolidone. The title compound was identified on the basis of its solid-state spectrum (see FIG. 8) and by MALDI-MS.

MALDI-MS: 503.051 [M+H]⁺, 489.045, 475.030.

### Example 5

### 5,5′-Diphenoxyindanthrone

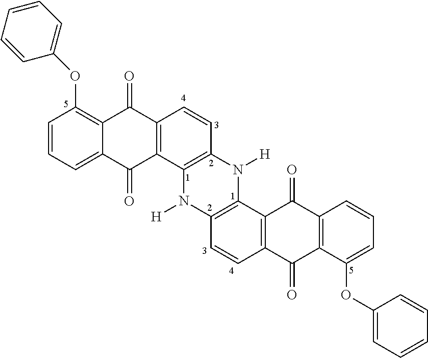

### 5.1 1-Phenoxy-5-nitroanthraquinone

A mixture of 10.0 g (33.5 mmol) of 1,5-dinitroanthraquinone, 3.16 g (33.5 mmol) of phenol and 27.8 g (201 mmol) of potassium carbonate in 200 ml of N-methylpyrrolidone was stirred at 40° C. for 2 hours. The reaction mixture was poured onto 5% sulfuric acid and stirred for one hour, and the precipitated beige precipitate was filtered off with suction, washed with water and dried. This gave 10.3 g (89%) of a crude product, which is purified by recrystallization. To this end, the crude product was dissolved in ethyl acetate, the residue was filtered off and the product was precipitated from the solution by adding petroleum ether. This gave 1.67 g (15%).

### 5.2 1-Phenoxy-5-aminoanthraquinone

The reduction was effected as described in example 4.2. The title compound was obtained in a yield of 91%.

### 5.3 5,5′-Diphenoxyindanthrone

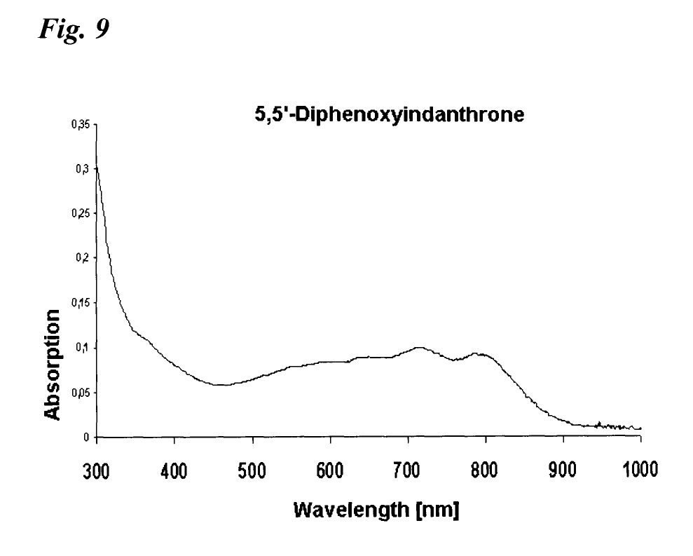

The title compound was synthesized as described in example 4.3. The title compound was purified by means of Soxhlet extraction from ethanol and chlorobenzene, and fractional crystallization from sulfuric acid. The title compound was identified on the basis of its solid-state spectrum (see FIG. 9) and by MALDI-MS.

MALDI-MS: 826.236, 649. 137 [M+Na]⁻, 627.156 [M+H]⁺, 611.158, 550.143, 455.329 and 441.309.

II) Performance Properties

Purification of Indanthrene Blue by Gradient Sublimation:

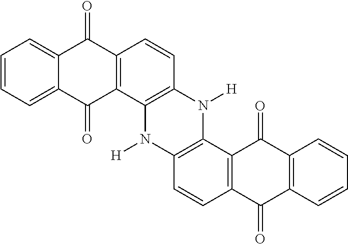

2.0 g of the material were purified by gradient sublimation. For this purpose, impurities were first removed by sublimation at 250° C./225° C./200° C. The sublimed impurities were removed and the resulting material was subjected to another sublimation at 350° C./325° C./300° C. This gave 1.1 g of product, which was sublimed again with the same temperature gradient (350° C./325° C./300° C.). This gave 916 mg of product, 500 mg of which were sublimed with the same temperature gradient (350° C./325° C./300° C.) and gave 420 mg of product. This material was used to produce the solar cells.

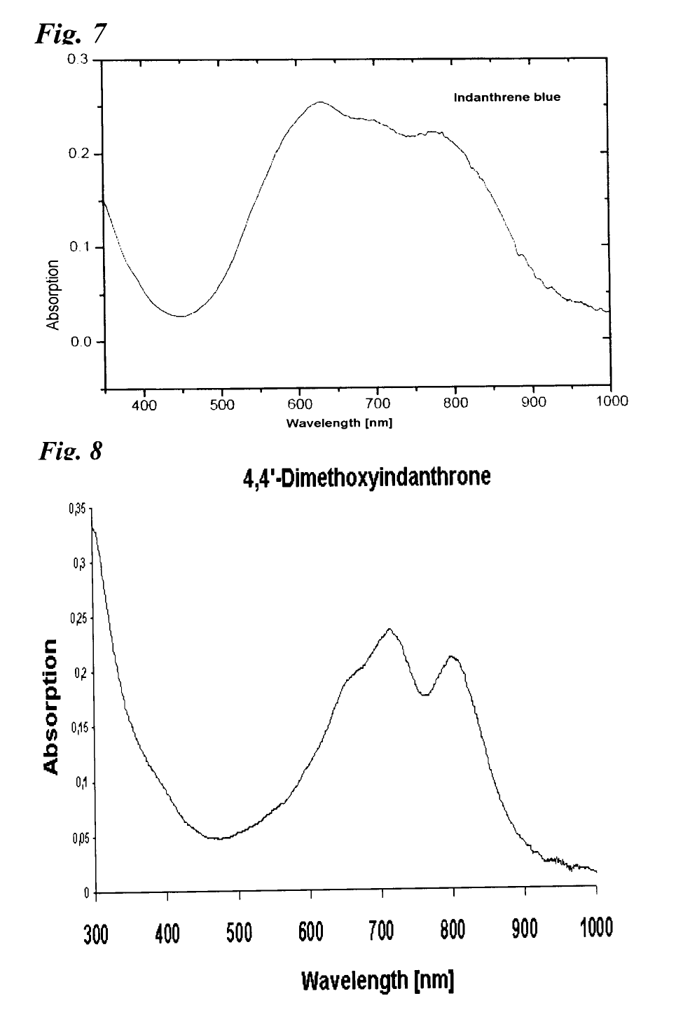

FIG. 7 shows the absorption spectrum of a vapor-deposited film of indanthrene blue. A long-wave absorption is observed, which is coupled with a good voltage V_{oc}.

Substrate:

ITO was sputtered onto the glass substrate in a thickness of 100 nm. The specific resistivity was 200 μΩcm, and the mean roughness (RMS; root mean square) was less than 5 nm. Before the deposition of the further layers, the substrate was treated with ozone under UV light for 20 minutes (UV-ozone cleaning).

Production of the Cells:

Bilayer cells (cells of two-layer construction) and bulk heterojunction cells (BHJ cells) were produced under high vacuum (pressure<10⁻⁶ mbar).

Bilayer Cell (ITO/indanthrene compound/C₆₀/Bphen/Ag):

The bilayer cell was produced by successive deposition of an indanthrene compound and C₆₀ onto the ITO substrate. The deposition rate for both layers was 0.1 nm/second. The evaporation temperatures of the indanthrene compound are reproduced in table 1 below.

TABLE 1

| | Evaporation temperature |
|---|---|
| Indanthrene compound | [° C.] |
| Indanthrene blue | 330 |
| Deuterated indanthrene blue (from example 1) | 200 |
| N,N′-Diphenylindanthrene (from example 2) | 240 |
| N,N′-Dimethylindanthrene (from example 3) | 200 |

C₆₀ was deposited at 410° C. Once the Bphen layer (layer thickness 6 nm) had been applied, a 100 nm-thick Ag layer was finally applied by vapor deposition as the top electrode. The cell had an area of 0.031 cm².

BHJ cell (ITO/(indanthrene compound:C₆₀−1:1 ratio by weight)/C₆₀/Bphen/Ag):

To produce the BHJ cell (bulk heterojunction cell), an indanthrene compound and the C₆₀ were coevaporated and applied to the ITO with the same deposition rate of 0.1 nm/second, such that there was a weight ratio of 1:1 in the mixed active layer. The Bphen and Ag layers were applied by vapor deposition as described for the bilayer cell. The layer thicknesses were 6 nm for BPhen and 100 nm for Ag.

Tests:

The solar simulator used was an AM 1.5 Simulator from Solar Light Co. Inc. with a xenon lamp (model 16S-150 V3). The UV range below 415 nm was filtered and the current-voltage measurements were made under ambient conditions. The intensity of the solar simulator was calibrated with a monocrystalline FZ solar cell (Fraunhofer ISE), and the deviation factor was determined to be approximately 1.0.

Results:

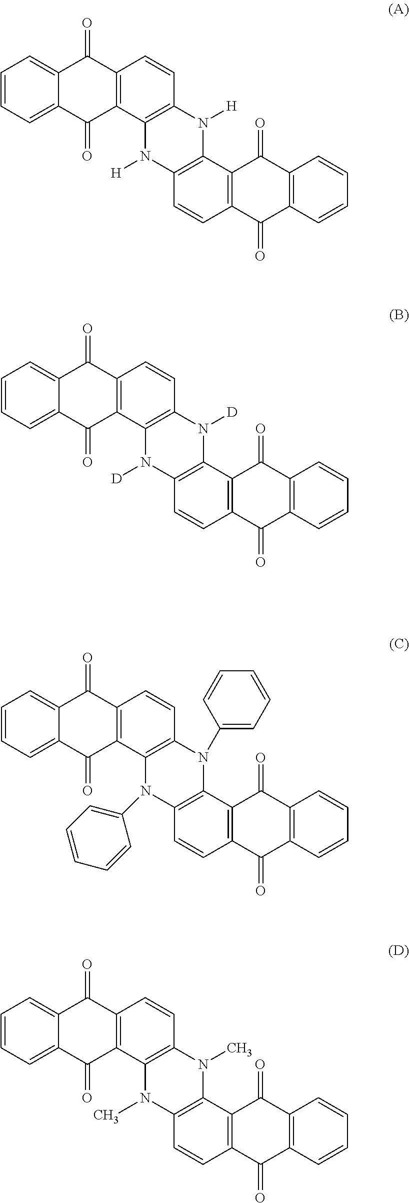

Bilayer Cell:

| | Layer | Layer | | | | |
|---|---|---|---|---|---|---|
| | thickness | thickness | V_{OC} | I_{SC} | FF | η |
| Compound | [nm] | [nm] | [mV] | [mA/cm²] | [%] | [%] |
| A | 20 | 40 | 660 | 2.1 | 58 | 0.8 |
| B | 20 | 40 | 660 | 2.6 | 56 | 0.9 |
| C | 20 | 40 | 660 | 2.4 | 27 | 0.4 |
| D | 10 | 40 | 630 | 2.5 | 36 | 0.6 |

BHJ Cell:

| | Layer | Layer | | | | |
|---|---|---|---|---|---|---|
| | thickness | thickness | V_{OC} | I_{SC} | FF | η |
| Compound | [nm] | [nm] | [mV] | [mA/cm²] | [%] | [%] |
| A | 10 | 20 | 700 | 5.1 | 45 | 1.6 |
| B | 30 | 20 | 740 | 5.9 | 42 | 1.8 |
| C | 10 | 20 | 740 | 3.2 | 48 | 1.1 |
| D | 10 | 20 | 580 | 3.7 | 43 | 0.9 |

η efficiency
FF fill factor
I_{sc} short-circuit current
V_{oc} open-circuit voltage

Cells comprising the combination of C60 with the indanthrene compounds A, B, C and D have high open-circuit voltages. In combination with a fullerene compound such as C60, the indanthrene compounds are suitable especially for use in tandem cells owing to their long-wave absorption.

1. An organic solar cell comprising at least one photoactive region which comprises at least one indanthrene compound which is in contact with at least one fullerene compound, wherein the indanthrene compound is selected from compounds of the general formula (I) 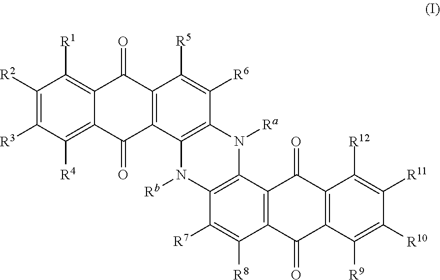
in which
R^{a} and R^{b} are each independently selected from hydrogen, deuterium, unsubstituted or substituted alkyl, unsubstituted or substituted cycloalkyl and unsubstituted or substituted aryl,
the R¹ to R¹² radicals are each independently selected from hydrogen, halogen, nitro, cyano, hydroxyl, carboxyl, carboxylate, SO₃H, sulfonate, Ne^{a}E^{b}, and in each case unsubstituted or substituted alkyl, alkoxy, alkylthio, cycloalkyl, aryl, aryloxy, arylthio, hetaryl, hetaryloxy, hetarylthio, oligo(het)aryl, oligo(het)aryloxy and oligo(het)alkylthio, where E^{a} and E^{b} are each independently hydrogen, alkyl, cycloalkyl or aryl.

2. The solar cell according to claim 1 in the form of a component cell of a tandem cell.

3. The organic solar cell according to claim 1, wherein, in the compounds of the general formula (I), the R^{a} and R^{b} radicals are each independently selected from hydrogen, deuterium, C₁-C₁₂-alkyl, C₇-C₂₂-aralkyl, C₄-C₇-cycloalkyl, C₆-C₁₀-aryl and C₇-C₂₂-alkaryl.

4. The organic solar cell according to any of the preceding claims, wherein, in the compounds of the general formula (I), the R^{a} and R^{b} radicals are both hydrogen or are both deuterium or are both C₁-C₆-alkyl or are both phenyl or are both C₁-C₁₂-alkylphenyl or are both naphthyl.

5. The organic solar cell according to any of the preceding claims, wherein, in the compounds of the general formula (I), the R¹ to R¹² radicals are each independently selected from hydrogen, F, Cl, hydroxyl, C₁-C₁₈-alkyl, C₁-C₁₂-alkoxy, C₁-C₆-alkylthio, C₇-C₂₂-aralkyl, C₇-C₂₂-aralkyloxy, C₇-C₂₂-aralkylthio, C₄-C₇-cycloalkyl, C₆-C₁₀-aryl, C₇-C₂₂-alkaryl, C₇-C₂₂-alkaryloxy, C₇-C₂₂-alkarylthio, amino, mono(C₁-C₁₂-alkyl)amino, di(C₁-C₁₂-alkyl)amino, NH(C₆-C₁₀-aryl), N(C₆-C₁₀-aryl)₂, hetaryl and oligohetaryl, where hetaryl and the hetaryl groups of oligohetaryl may each independently be unsubstituted or substituted by 1, 2, 3 or 4 radicals selected from C₁-C₁₂-alkyl and C₁-C₁₂-alkoxy.

6. The organic solar cell according to any of the preceding claims, wherein, in the compounds of the general formula (I), the R¹ to R¹² radicals are each independently selected from hydrogen, C₁-C₁₂-alkyl, C₁-C₁₂-alkoxy, phenyl, naphthyl, phenyloxy, naphthyloxy and oligothiophenyl, where phenyl, naphthyl, phenyloxy, naphthyloxy and oligothiophenyl are unsubstituted or have 1 or 2 substituents which are selected from C₁-C₁₂-alkyl and C₁-C₁₂-alkoxy.

7. The organic solar cell according to any of the preceding claims, wherein, in the compounds of the general formula (I), 0, 1, 2, 3 or 4 of the R¹ to R¹² radicals have a definition other than hydrogen.

8. The organic solar cell according to any of the preceding claims, wherein the indanthrene compound is selected from compounds of the general formula (I.1) 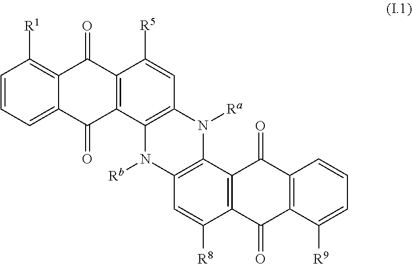
where
R^{a} and R^{b} are each independently selected from hydrogen, deuterium,
C₁-C₆-alkyl, phenyl and naphthyl,
R¹ and R⁹ are each independently selected from phenyl, phenyloxy, phenylthio, naphthyl, naphthyloxy, naphthylthio, (C₁-C₁₂-alkyl)phenyl, (C₁-C₁₂-alkyl)phenyloxy, (C₁-C₁₂-alkyl)phenylthio, (C₁-C₁₂-alkyl)naphthyl, (C₁-C₁₂-alkyl)naphthyloxy and (C₁-C₁₂-alkyl)naphthylthio,
R⁵ and R⁸ are each independently selected from hydrogen, hydroxyl and C₁-C₁₂-alkoxy.

9. The organic solar cell according to any of the preceding claims, wherein the photoactive region comprises, as the fullerene compound, at least one fullerene and/or fullerene derivative.

10. The organic solar cell according to claim 8, wherein the photoactive region comprises, as the fullerene compound, C60 or [6,6]-phenyl-C61-butyric acid methyl ester.

11. The organic solar cell according to any of the preceding claims, wherein at least one photoactive donor-acceptor transition is present in the form of a bulk heterojunction.

12. The use of a compound of the general formula (I) as defined in any of claims 1 to 7 as an electron donor in organic photovoltaics.

13. The use of a compound of the general formula (I) as defined in any of claims 1 to 7 as a photoactive material in an organic solar cell.

14. The use according to claim 13, in an organic solar cell which comprises at least one photoactive region which comprises at least one compound of the general formula (I) as defined in any of claims 1 to 7 and at least one fullerene compound.

15. The use according to claim 14, wherein the fullerene compound used is C60 or [6,6]-phenyl-C61-butyric acid methyl ester.

16. The use according to any of claims 12 to 15 in a tandem cell.

17. A compound of the formula

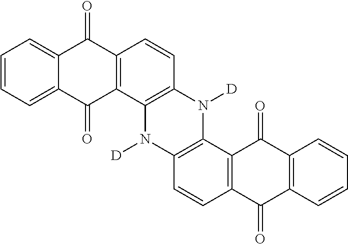

18. A compound of the formula I 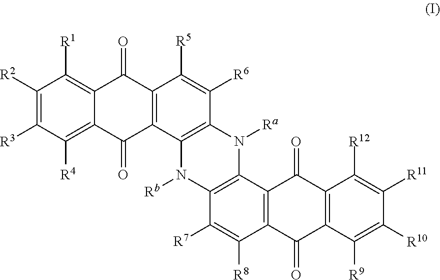
in which R¹ and R⁹ are both phenoxy, and the R^{a}, R^{b}, R², R³, R⁴, R⁵, R⁶, R⁷, R⁸, R¹⁰, R¹¹ and R¹² radicals are all hydrogen; or
R⁵ and R⁸ are both methoxy and the R^{a}, R^{b}, R¹, R², R³, R⁴, R⁶, R⁷, R⁹, R¹⁰, R¹¹ and R¹² radicals are all hydrogen.
